(19) United States
(12) Patent Application Publication
(10) Pub. No.: US 2026/0263559 A1
(43) Pub. Date: Sep. 10, 2026

(54) RECOMBINANT PROTEIN COMPOSITIONS USEFUL FOR WOUND HEALING

(71) Applicant: Wake Forest University Health Sciences, Winston-Salem, NC (US)

(72) Inventors: Ji Hyun KIM, Winston-Salem, NC (US); Young Min JU, Pfafftown, NC (US); Sang Jin LEE, Clemmons, NC (US); Bill VAUGHAN, Greensboro, NC (US); John D. JACKSON, Clemmons, NC (US); James J. YOO, Winston-Salem, NC (US); Anthony ATALA, Winston-Salem, NC (US)

(21) Appl. No.: 19/167,113

(22) PCT Filed: Mar. 20, 2024
(86) PCT No.: PCT/US2024/020633

§ 371 Date: Sep. 19, 2025

Related U.S. Application Data
(60) Provisional application No. 63/453,784, filed on Mar. 22, 2023.

(51) Int. Cl. A61K 38/18; A61K 9/06; A61K 9/12; A61K 38/17; A61K 38/20; A61K 38/28; A61K 47/36; A61K 47/42; A61P 17/02

(52) U.S. Cl. CPC A61K 38/1825; A61K 9/06; A61K 9/122; A61K 38/1774; A61K 38/18; A61K 38/1833; A61K 38/1841; A61K 38/1866; A61K 38/204; A61K 38/28; A61K 47/36; A61K 47/42; A61P 17/02

(57) ABSTRACT

Described herein are compositions comprising a combination of active agents, such as recombinant fibroblast growth factor-7 (FGF-7), recombinant follistatin, recombinant interleukin-6 (IL-6), recombinant transforming growth factor beta-1 (TGFβ-1), recombinant urokinase receptor (uPAR), and a carrier, which compositions may further comprise recombinant angiopoietin-1 (ANG-1), recombinant hepatocyte growth factor (HGF), recombinant insulin, and/or recombinant vascular endothelial growth factor (VEGF). Also provided herein are methods of treating wounds, accelerating healing of a wound, accelerating angiogenesis, and/or reducing scarring, as well as methods for culturing cells in vitro, and for cell delivery in vivo, using compositions of the present invention.

### CROSS REFERENCE TO RELATED APPLICATIONS

This application claims the benefit of and priority to U.S. Provisional Patent Application Ser. No. 63/453,784 filed Mar. 22, 2023, the disclosure of which is incorporated herein by reference in its entirety.

### BACKGROUND

Extensive burns and full-thickness skin wounds can be devastating to patients, even when treated. In addition to burns, full-thickness chronic wounds constitute a large patient base, and despite technological advancement of treatments, successful healing rates remain below 50% (Kurd et al., Wound Repair Regen 2009; 17:318-325). Patients who suffer from either of these types of injuries benefit from rapid treatments that result in complete closure and protection of the wounds. In particular, burn patients who receive delayed treatments are often subject to extensive scarring that can result in negative long-term physiological effects.

The use of spilt thickness autografts is currently the gold standard of treatment, but this option is often limited by the availability of a suitable area of healthy donor skin to harvest (Sen et al., Wound Repair Regen 2009; 17:763-771). Cellular and non-cellular biological skin-equivalents may be used as an alternative treatment option for these patients; however, these treatments usually involve multiple surgical procedures and the associated high costs for production and repeated wound treatment.

Cellular therapy is a promising alternative to biological skin-equivalents because a successful cell-based technique could rapidly cover wounds and accelerate healing using living components. Fetal stem cells possess the ability to regenerate and restore normal tissue function in multiple tissue and organ systems, including skin, kidney and corpora, and application of fetal stem cells to injury models facilitates rapid regeneration and recovery. Studies indicate that fetal stem cells secrete factors at concentrations higher than those of mesenchymal stem cells (MSC) and that fetal stem cell-conditioned media induces endothelial cell migration in vitro (Skardal et al., Bioprinted amniotic fluid-derived stem cells accelerate healing of large skin wounds. Stem Cells Transl Med. 2012 November; 1(11):792-802). These secretomes, which denote all the proteins secreted by a cell, along with the secretory pathway constituents, include growth factors, chemokines, cytokines, adhesion molecules, proteases and shed receptors.

Stem cell-derived secretomes can enhance tissue regeneration, and conditioned media (CM) derived from fetal stem cells have similar therapeutic effects as stem cell transplantation in restoring normal tissue function (Pawitan, BioMed research international. 2014 (2014) 965849). However, stem cell-derived secretomes as a treatment modality is challenged by safety concerns due to the undefined biomolecules found in CM.

European Patent application publication EP 2916876 to Agarwal et al. relates to methods and compositions for wound healing by utilizing cross-linker covalent modification molecules to attach and deliver wound active agents to a wound, and further relates to incorporating wound active agents into a polyelectrolyte layer for delivery to a wound. However, particular active agent compositions are not disclosed.

U.S. Pat. No. 8,728,498 to Zhang et al. relates to preparing silk fibroin/polyethylene oxide blended materials suitable for biomedical applications such as wound healing, which may include therapeutic agents embedded therein. However, particular combinations of therapeutic agents are not disclosed.

U.S. Pat. No. 8,796,025 to Emig et al. is directed to methods for the treatment of wounds with amnion-derived multipotent cells, or conditioned media or other cell products derived therefrom, which may include secreted cytokines. However, many different cytokines were detected in the conditioned media samples.

Improved options are needed for providing compositions useful to promote wound healing.

### SUMMARY

Compositions in accordance with the present invention may include (comprise, consist of or consist essentially of) particular active agents (or “factors”) found in conditioned media, such as: fibroblast growth factor-7 (FGF-7), follistatin, interleukin-6 (IL-6), transforming growth factor beta-1(TGFβ-1), and/or urokinase receptor (uPAR). Compositions may also include: angiopoietin-1 (ANG-1), hepatocyte growth factor (HGF), insulin, and/or vascular endothelial growth factor (VEGF).

Compositions provided herein according to some embodiments may include:
i) from 0.5 or 1 ng/mL, to 4 or 5 ng/mL of recombinant fibroblast growth factor-7 (FGF-7);
ii) from 5, 6 or 8 ng/mL, to 28 or 30 ng/mL of recombinant follistatin;
iii) from 2, 3 or 4 ng/mL to 14 or 16 ng/mL of recombinant interleukin-6 (IL-6);
iv) from 15, 20 or 25 ng/mL to 110 or 125 ng/mL of recombinant transforming growth factor beta-1 (TGFβ-1); and
v) from 2 or 3 ng/mL, to 14 or 16 ng/mL of recombinant urokinase receptor (uPAR); and a carrier. In some embodiments, the composition further includes:
vi) from 2 or 3 ng/mL, to 11 or 12 ng/mL of recombinant angiopoietin-1 (ANG-1);
vii) from 6, 7, 8 or 9 ng/mL, to 45 or 50 ng/mL of recombinant hepatocyte growth factor (HGF);
viii) from 6, 7, 8 or 9 ng/mL, to 45 or 50 ng/mL of recombinant insulin; and
ix) from 1 or 1.5 ng/mL, to 6, 8 or 10 ng/mL of recombinant vascular endothelial growth factor (VEGF).

In some embodiments, the composition includes:
i) from 1 to 3 ng/mL of recombinant fibroblast growth factor-7 (FGF-7);
ii) from 8 to 25 ng/mL of recombinant follistatin;
iii) from 4 to 12 ng/mL of recombinant interleukin-6 (IL-6);
iv) from 25 to 100 ng/mL of recombinant transforming growth factor beta-1 (TGFβ-1); and
v) from 3 to 12 ng/mL of recombinant urokinase receptor (uPAR). In some embodiments, the composition further includes:
vi) from 3 to 10 ng/mL of recombinant angiopoietin-1 (ANG-1);
vii) from 9 to 40 ng/mL of recombinant hepatocyte growth factor (HGF);
viii) from 9 to 40 ng/mL of recombinant insulin; and
ix) from 1.5 to 5 ng/mL of recombinant vascular endothelial growth factor (VEGF).

In some embodiments, the composition includes:
i) from 1.5 to 3 ng/mL of recombinant FGF-7;
ii) from 12 to 25 ng/mL of recombinant follistatin;
iii) from 6 to 12 ng/mL of recombinant IL-6;
iv) from 45 to 100 ng/mL of recombinant TGFβ-1; and
v) from 6 to 12 ng/mL of recombinant uPAR. In some embodiments, the composition further includes:
vi) from 5 to 10 ng/mL of recombinant ANG-1;
vii) from 18 to 40 ng/mL of recombinant HGF;
viii) from 18 to 40 ng/mL of recombinant insulin; and
ix) from 3 to 5 ng/mL of recombinant VEGF.

In some embodiments, the composition includes:
i) from 1.5 to 2.5 ng/mL of recombinant FGF-7;
ii) from 12 to 20 ng/mL of recombinant follistatin;
iii) from 6 to 10 ng/mL of recombinant IL-6;
iv) from 45 to 80 ng/mL of recombinant TGFβ-1; and
v) from 6 to 10 ng/mL of recombinant uPAR. In some embodiments, the composition further includes:
vi) from 5 to 8 ng/mL of recombinant ANG-1;
vii) from 18 to 30 ng/mL of recombinant HGF;
viii) from 18 to 30 ng/mL of recombinant insulin; and
ix) from 3 to 4 ng/mL of recombinant VEGF.

In some embodiments, the composition includes:
i) from 1.5 ng/mL to 6, 10, 15, 18 or 21 ng/mL of recombinant FGF-7;
ii) from 12 ng/mL to 35, 50, 75, 100, 125, 150, or 165 ng/mL of recombinant follistatin;
iii) from 6 ng/mL to 20, 35, 50, 70, or 80 ng/mL of recombinant IL-6;
iv) from 45 ng/mL to 150, 200, 300, 500, or 620 ng/mL of recombinant TGFβ-1; and/or
v) from 6 ng/mL to 20, 35, 50, 65 or 78 ng/mL of recombinant uPAR. In some embodiments, the composition further includes:
vi) from 5 ng/mL to 15, 20, 30, 45, or 63 ng/mL of recombinant ANG-1;
vii) from 18 ng/mL to 75, 100, 125, 150, 200, or 250 ng/mL of recombinant HGF;
viii) from 18 ng/mL to 75, 100, 125, 150, 200, or 250 ng/mL of recombinant insulin; and/or
ix) from 3 ng/mL to 12, 15, 20, 30 or 35 ng/mL of recombinant VEGF.

In some embodiments, the carrier comprises a media or saline. In some embodiments, the carrier is sterile. In some embodiments, the composition is frozen (e.g., freeze-dried). In some embodiments, the carrier is a hydrogel. In some embodiments, the carrier is a foam. In some embodiments, the carrier comprises a cream or gel suitable for topical application to a skin wound. In some embodiments, the carrier is in the form of a patch or sheet.

In some embodiments, the hydrogel or foam comprises alginate (e.g., crosslinked alginate), gelatin, or a combination thereof (e.g., a combination thereof at a ratio of from 2:1 or 1.5:1, to 1:1.5 or 1:2 by weight) (e.g., 1, 2 or 3% by weight of an alginate and gelatin mixture).

Also provided is a method for treating a wound, comprising applying a composition as taught herein to said wound. In some embodiments, the composition is applied to said wound in an amount effective to accelerate healing of the wound (e.g., accelerate wound closure, tissue formation, and/or re-epithelialization). In some embodiments, the composition is applied to said wound in an amount effective to accelerate angiogenesis of the wound. In some embodiments, the composition is applied to said wound in an amount effective to reduce scarring (e.g., promote basket-weave collage network formation).

In some embodiments, the wound is hypoxic. In some embodiments, the wound is a burn wound. In some embodiments, the wound is a full-thickness skin wound (e.g., full-thickness chronic skin wound).

Further provided is a method for culturing cells in vitro, comprising culturing said cells in the presence of a composition as taught herein. In some embodiments, the composition is provided in an amount effective to promote proliferation of the cells.

Still further provided is a method for cell delivery in vivo, comprising providing a composition as taught herein which contains said cells, and delivering the composition to a subject in need thereof. Also provided is the use of a composition as taught herein in a method for cell delivery in vivo, or for preparing a medicament for cell delivery in vivo.

### BRIEF DESCRIPTION OF THE DRAWINGS

FIG. 1. Human dermal fibroblasts (HDF) were treated with serum-free medium (SFM) (conditioned medium factors (CMF)0×) and various CMF (nine protein combination: ANG-1, FGF-7, Follistatin, HGF, IL-6, Insulin, TGFβ-1, uPAR, and VEGF) concentrations (CMF 1×, 2×, 4×, 6×, 8×, and 10×, Table 2), and cell growth measured using Alamar Blue assay. Panels A,B: Cell number cultured in normoxia (A) and hypoxia (B). Panels C,D: Linear regression relation between CMF concentration and cell number in normoxia (C) and hypoxia (D). In hypoxia, CMF 10× data is excluded. The results indicate that CMF increases cell growth in a dose-dependent manner, but not in concentrations greater than 6×. Overall, CMF at a concentration of 6× is the optimal concentration to enhance HDF growth (n=3, duplicate measure, two-way ANOVA and Tukey's test, (A) *p<0.05 with SFM, †p<0.05 with CMF 1×, ‡p<0.05 with CMF 2×, 4×, and 6×, NS: p>0.05, (B) *p<0.05 with SFM. †p<0.05 with CMF 1×. ‡p<0.05 with CMF 2×, § p<0.05 with CMF 4×, 6×, and 8×, NS: p>0.05).

FIG. 2. The effect of the recombinant protein combination (CMF) on HDF growth compared with individual proteins. Treatment with individual proteins (ANG-1, FGF-7, Follistatin, HGF, IL-6, Insulin, TGFβ-1, uPAR, or VEGF) or their combination (CMF-ANG-1, FGF-7, Follistatin, HGF, IL-6, Insulin, TGFβ-1, uPAR, and VEGF) for 0-, 2-, and 5-day culture under normoxic (A) and hypoxic (B) conditions. The cell number of each group is measured using AlamarBlue assay and normalized to the day 0 cell number. The CMF shows significantly increased cell number compared with all individual proteins (†p<0.05) at day 2 and day 5 in normoxia and hypoxia (n=3, duplicate measure, two-way ANOVA and Tukey's test, *p<0.05 with SFM, †p<0.05 with ANG-1, FGF-7, Follistatin, HGF, IL-6, Insulin, TGFβ-1, uPAR, and VEGF).

FIG. 3. The efficacy of CMF compared with CM and human placenta stem cell (hPSC) co-culture. HDF is treated with growth medium (GM), SFM, CM(6×), CMF (6×), and hPSC (co-culture with hPSC) for 0, 2, and 5 days in normoxia (A) and hypoxia (B). Normalized cell numbers are measured using AlamarBlue assay. CMF has a similar effect to CM and co-culture treatment on HDF growth (n=3, duplicate measure, two-way ANOVA and Tukey's test, *p<0.05 with SFM, †p<0.05 with CMF, ‡p>0.05 with GM).

FIG. 4. HDF proliferation and anti-apoptosis effect of CMF treatment. HDF is treated with GM, SFM, CM (6×), and CMF (6×) for 3 days, and pro-proliferative and anti-apoptotic effect is investigated using Proliferating cell nuclear antigen (PCNA) and Cleaved caspase-3 (CC3) ELISA assay. Panels A,B: PCNA (ng/mg) in normoxia (A) and hypoxia (B). Panels C,D: CC3 (pg/mg) in normoxia (C) and hypoxia (D). PCNA protein levels show no significant difference between GM, SFM, CM, and CMF. CMF and CM prevent HDF apoptosis but do not improve proliferation significantly (n=3, one-way ANOVA and Tukey's test, * p<0.05 with SFM, †p>0.05 with GM).

FIG. 5. In Vitro wound healing assay. HDF migration to the wound gap after 1 day of treatment with GM, SFM, CM (6×), and CMF (6×) in normoxia and hypoxia. (A) Representative images of Cytoselect cell staining. (B) Cell Migration (%). CMF accelerates cell migration and wound closure (n=4 per group, 6 random fields per sample, one-way ANOVA and Tukey's test, *p<0.05 with SFM. †p>0.05 with GM). Dashed lines indicate the initial wound gap (0.9 mm). Scale bars: 0.9 mm.

FIG. 6. Angiogenic properties of CMF in in ovo chick chorioallantoic membrane (CAM) assay. Gelfoam® scaffold treated with CMF (6×) and Control (PBS) is implanted on CAM, incubated in ovo for 5 days, and blood vessel formation is evaluated. (A) Brightfield images of CAM model at day 0 and day 5. (B) Quantitative analysis of newly formed blood vessel peripheral to scaffold after 5 days implantation (n=6 per group, T-Test, and Welch's correction *p<0.05 with control). (C) Histological analysis of vertical and horizontal sections through CAM. Upper panel: H&E (Scale bars: 50 μm) of vertical sections. Bottom panel: H&E (Scale bars: 200 μm) of horizontal sections. (D) Quantitative analysis of blood vessels per H&E day 5 images (n=4 per group, T-Test, and Welch's correction *p<0.05 with control).

FIG. 7. The efficacy of CMF compared with CM and human placenta stem cell (hPSC) co-culture on human umbilical vein endothelial cell (HUVEC) growth. HUVECs are cultured in normoxia (A) and hypoxia (B) with HUVEC-GM, HUVEC-SFM, hPSC-CM 6×, CMF 6×, and hPSC-HUVEC co-culture. Cell number is calculated using alamarBlue assay and normalized to the cell number at day 0. HUVEC number treated with CMF is significantly higher than SFM but lower than CM and co-culture (n=4, duplicate measure, two-way ANOVA and Tukey's test, *p<0.05 with SFM, †p<0.05 with CMF).

FIG. 8. Alginate/gelatin composite hydrogel system. (A) Optical photographs of the alginate-gelatin composite solution and crosslinked hydrogel by applying with 10 mM of CaCl₂ (100 mM). (B) Water uptake property of the alginate-only hydrogel and the alginate/gelatin hydrogel (n=5, *p<0.05 with alginate-only). (C) Weight changes of the alginate-only hydrogel and the alginate/gelatin hydrogel for each of the time points (n=5). (D) In vitro release of FITC-dextran (20 kDa. and 70 kDa.) from the alginate/gelatin hydrogel. Cumulative % release of FITC-dextran at pH 7.4 and 37° C. for each of the time points up to 14 days (n=5). Dex: dextran.

FIG. 9. Physical properties of the alginate combined with various hydrogels. (A) Water uptake property (n=5, *p<0.05 with alginate-only). (B) Weight changes for each of the time points (n=5).

FIG. 10. In vitro biological evaluations of the alginate/gelatin hydrogel. (A) Representative Live/Dead staining images (×100 magnification; scale bar 100 μm) HPSCs for cytotoxic evaluation. ALG: alginate (B) Cell viability was measured using alamarBlue metabolic activity assay (n=5; *p<0.05 with positive control). N: negative, P: positive. (C) HPSCs proliferation in the hydrogels (n=5; *p<0.05 with alginate only at Day 3, **p<0.05 with alginate-only at Day 7). Cell proliferation rate was assessed by CellTiter-Glo 3D assay.

FIG. 11. Biologic activity of the alginate-based various hydrogels in vitro. (A) Representative Live/Dead staining images (100× magnification; scale bar 100 m) for cytotoxic evaluation of the HDFs. (B) HDFs viability was measured using alamarBlue metabolic activity assay (n=5; *p<0.05 with positive control). (C) Representative Live/Dead staining images (100× magnification; scale bar 100 μm) for cytotoxic evaluation of the HPSCs. (D) HPSCs viability was measured using alamarBlue metabolic activity assay (n=5; *p<0.05 with positive control). (E) HPSCs proliferation of the cell-encapsulated hydrogels (n=5; *p<0.05 with alginate only at Day 3, **p<0.05 with alginate-only at Day 7). Cell proliferation rate was assessed by CellTiter-Glo 3D assay.

FIG. 12. A percentage of the original wound area was calculated by subtracting the open wound area from the original wound area. Open wound area was measured for each treatment group at each timepoint using image J software. (n=5, *p<0.05 with all other groups)

FIG. 13. Representative histological images for Hematoxylin and Eosin (H&E). (A) Representative H&E-stained histological images (100× magnification; scale bar 100 μm). (B) Summary table of wound healing assessed by cellularity, tissue granulation, epidermal regeneration, angiogenesis, and hair follicle formation.

FIG. 14. Representative histological images for Masson's Trichrome. (A) Representative Masson's Trichrome stained histological images (100× magnification; scale bar 100 μm). (B) Summary table of re-epithelialization index assessed by the thickness of Keratin tissue formation.

FIG. 15. Representative histological images for Picrosirius Red. (A) Representative Picrosirius Red stained histological images under polarizing light (400× magnification; scale bar 20 μm). Native skin is dominated by mature collagen tissue in a basket weave orientation. (B) Collagen fiber width was measured using CT-Fire software and is represented as a length in pixels (NS, p>0.05; n>200 fibers per group). (C) Collagen fiber width was measured using CT-Fire software and is represented as a length in pixels (NS, p>0.05; n>200 fibers per group). (D) Collagen fiber alignment was measured using CurveAlign software, and is represented as alignment, with zero being no alignment or more normal, and 1 being complete alignment or more scar-like tissue (*p<0.05 with native; NS, p>0.05; n>200 fibers per group).

FIG. 16. Representative immunofluorescent images for α-Smooth Muscle Actin (αSMA). (A) Representative images of αSMA immunofluorescent images (200× magnification; scale bare 50 μm). (B) Number of vessel counts and (C) SMA+vessel diameter were measured for each treatment group at each timepoint using image J software (n>10, *, **, +, ++p<0.05 with defect only control group).

FIG. 17. Representative immunofluorescent images for Ki67. (A) Representative images of Ki67 immunofluorescent images (200× magnification; scale bare 50 m). (B) Number of Ki67+cell counts were measured for each treatment group at each timepoint using image J software (n>10, *, **, *** p<0.05 with defect only control group).

FIG. 18. Schematic of an example preparation of an alginate-gelatin hydrogel and foam.

FIG. 19. Cumulative release profile of FITC-dextran with both 20 kDa and 70 kDa molecular weights over 14 days in the alginate-gelatin hydrogel (“Gel”) and foam.

FIG. 20. CMF efficacy in vitro (HDF), showing cell proliferation on day 2 and day 5 with gel control (no CMF), gel+CMF, and foam+CMF.

FIG. 21. CMF stability test in vitro (HDF), with carriers stored 1, 2, 3, or 4 weeks at different storage temperatures.

### DETAILED DESCRIPTION

The present invention is now described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” As a specific example, a composition that consists essentially of particular CM factors as taught herein should be interpreted as not including other CM factors not listed.

The term “about,” as used herein when referring to a measurable value such as an amount or concentration and the like, is meant to encompass variations of ±10%, 5%, 1%, 0.5%, or even±0.1% of the specified value as well as the specified value. For example, “about X” where X is the measurable value, is meant to include X as well as variations of ±10%, 5%, 1%, 0.5%, or even±0.1% of X. A range provided herein for a measurable value may include any other range and/or individual value therein.

“Treat” as used herein refers to any type of treatment that imparts a benefit to a subject or patient, and may include, but is not limited to, an improvement of wound healing.

“Subjects” are generally human subjects and include, but are not limited to, “patients.” The subjects may be male or female and may be of any age, including newborn, neonate, infant, child, adolescent, adult, and geriatric.

Subjects may also include animal subjects, particularly mammalian subjects such as canines, felines, bovines, caprines, equines, ovines, porcines, rodents (e.g., rats and mice), lagomorphs, primates (including non-human primates), etc., for, e.g., veterinary medicine and/or pharmaceutical drug development purposes.

### 1. Compositions

Compositions useful for promoting cell proliferation and/or wound healing may include (comprise, consist of or consist essentially of) combinations of active agents as taught herein. In some embodiments, the active agents are recombinant proteins. “Recombinant” protein as used herein refers to a protein encoded by and produced with the use of an expression system introduced into a host cell (e.g., a eukaryotic host cell). Such proteins are typically collected and purified and can be concentrated to produce a stock concentration.

Compositions in accordance with the present invention may include particular active agents (also called “factors” herein) found in conditioned media, such as (but not limited to): fibroblast growth factor-7 (FGF-7), follistatin, interleukin-6 (IL-6), transforming growth factor beta-1 (TGFβ-1), and/or urokinase receptor (uPAR). Compositions may also include (but not limited to): angiopoietin-1 (ANG-1), hepatocyte growth factor (HGF), insulin, and/or vascular endothelial growth factor (VEGF).

In some embodiments, the composition may include:
i) from 0.5 or 1 ng/mL, to 4 or 5 ng/mL of recombinant fibroblast growth factor-7 (FGF-7);
ii) from 5, 6 or 8 ng/mL, to 28 or 30 ng/mL of recombinant follistatin;
iii) from 2, 3 or 4 ng/mL to 14 or 16 ng/mL of recombinant interleukin-6 (IL-6);
iv) from 15, 20 or 25 ng/mL to 110 or 125 ng/mL of recombinant transforming growth factor beta-1 (TGFβ-1); and
v) from 2 or 3 ng/mL, to 14 or 16 ng/mL of recombinant urokinase receptor (uPAR).

In some embodiments, the composition may further include:
vi) from 2 or 3 ng/mL, to 11 or 12 ng/mL of recombinant angiopoietin-1 (ANG-1);
vii) from 6, 7, 8 or 9 ng/mL, to 45 or 50 ng/mL of recombinant hepatocyte growth factor (HGF);
viii) from 6, 7, 8 or 9 ng/mL, to 45 or 50 ng/mL of recombinant insulin; and
ix) from 1 or 1.5 ng/mL, to 6, 8 or 10 ng/mL of recombinant vascular endothelial growth factor (VEGF).

In some embodiments, composition may include:
i) from 1 to 3 ng/mL of recombinant fibroblast growth factor-7 (FGF-7);
ii) from 8 to 25 ng/mL of recombinant follistatin;
iii) from 4 to 12 ng/mL of recombinant interleukin-6 (IL-6);
iv) from 25 to 100 ng/mL of recombinant transforming growth factor beta-1 (TGFβ-1); and/or
v) from 3 to 12 ng/mL of recombinant urokinase receptor (uPAR).

In some embodiments, composition may further include:
vi) from 3 to 10 ng/mL of recombinant angiopoietin-1 (ANG-1);
vii) from 9 to 40 ng/mL of recombinant hepatocyte growth factor (HGF);
viii) from 9 to 40 ng/mL of recombinant insulin; and/or
ix) from 1.5 to 5 ng/mL of recombinant vascular endothelial growth factor (VEGF).

In some embodiments, the composition may include:
i) from 1.5 to 3 ng/mL of recombinant FGF-7;
ii) from 12 to 25 ng/mL of recombinant follistatin;
iii) from 6 to 12 ng/mL of recombinant IL-6;
iv) from 45 to 100 ng/mL of recombinant TGFβ-1; and/or
v) from 6 to 12 ng/mL of recombinant uPAR.

In some embodiments, the composition may further include:
vi) from 5 to 10 ng/mL of recombinant ANG-1;
vii) from 18 to 40 ng/mL of recombinant HGF;
viii) from 18 to 40 ng/mL of recombinant insulin; and/or
ix) from 3 to 5 ng/mL of recombinant VEGF.

In some embodiments, the composition may include:
i) from 1.5 to 2.5 ng/mL of recombinant FGF-7;
ii) from 12 to 20 ng/mL of recombinant follistatin;
iii) from 6 to 10 ng/mL of recombinant IL-6;
iv) from 45 to 80 ng/mL of recombinant TGFβ-1; and/or
v) from 6 to 10 ng/mL of recombinant uPAR.

In some embodiments, the composition may further include:
vi) from 5 to 8 ng/mL of recombinant ANG-1;
vii) from 18 to 30 ng/mL of recombinant HGF;
viii) from 18 to 30 ng/mL of recombinant insulin; and/or
ix) from 3 to 4 ng/mL of recombinant VEGF.

In some embodiments, the composition may include:
i) from 1.5 ng/mL to 6, 10, 15, 18 or 21 ng/mL of recombinant FGF-7;
ii) from 12 ng/mL to 35, 50, 75, 100, 125, 150, or 165 ng/mL of recombinant follistatin;
iii) from 6 ng/mL to 20, 35, 50, 70, or 80 ng/mL of recombinant IL-6;
iv) from 45 ng/mL to 150, 200, 300, 500, or 620 ng/mL of recombinant TGFβ-1; and/or
v) from 6 ng/mL to 20, 35, 50, 65 or 78 ng/mL of recombinant uPAR.

In some embodiments, the composition may further include:
vi) from 5 ng/mL to 15, 20, 30, 45, or 63 ng/mL of recombinant ANG-1;
vii) from 18 ng/mL to 75, 100, 125, 150, 200, or 250 ng/mL of recombinant HGF;
viii) from 18 ng/mL to 75, 100, 125, 150, 200, or 250 ng/mL of recombinant insulin; and/or
ix) from 3 ng/mL to 12, 15, 20, 30 or 35 ng/mL of recombinant VEGF.

The combinations of active agents as taught herein may be provided in a suitable carrier, such as a pharmaceutically acceptable carrier. “Pharmaceutically acceptable” as used herein means that the carrier is suitable for administration to a subject, without unduly deleterious side effects in light of the severity of the subject's condition and necessity of the treatment.

In some embodiments, the carrier is aqueous, such as, for example, water, saline, or media. In some embodiments, the carrier is sterile. Non-limiting examples of an aqueous sterile carrier include endotoxin-free or pyrogen-free water or saline.

In some embodiments, the carrier is or comprises a hydrogel. “Hydrogel” as used herein refers to naturally-derived hydrogels, synthetic hydrogels, or a combination thereof. Naturally-derived hydrogels may include, but are not limited to, collagen, chitosan, hyaluronic acid, gelatin, gellan gum, alginate, fibrin, or mixtures of two or more thereof. In some embodiments, the hydrogel comprises alginate in combination with another naturally-derived hydrogel, such as gelatin or gellan gum.

Naturally-derived hydrogels in some embodiments may include decellularized tissue extracts. Extracellular matrix may be collected from a specific tissue and may be combined with a hydrogel material to be used to support cells of that tissue type. See, e.g., Skardal et al., Tissue Specific Synthetic ECM Hydrogels for 3-D in vitro Maintenance of Hepatocyte Function, Biomaterials 33 (18): 4565-75 (2012).

Synthetic hydrogels may be produced from a variety of materials (e.g., poly-(ethylene glycol) (PEG), poly(vinyl alcohol) (PVA), etc.) and using many techniques. In contrast to many naturally-derived hydrogels, synthetic hydrogels may be produced uniformly and may be easily reproducible and characterized. Synthetic hydrogels may, however, lack some functional signals for cells, like the active sites found in natural extracellular matrix, limiting their potential to support cells. See, e.g., Mahoney et al., Three-Dimensional Growth and Function of Neural Tissue in Degradable Polyethylene Glycol Hydrogels, Biomaterials 27 (10): 2265-74 (2006). Combined hydrogels may offer a compromise and may allow for more control over the ability to reconstruct a specific microenvironment. By combining natural components, such as extracellular matrix molecules (e.g., extracellular matrix proteins), with defined synthetic hydrogels, a more easily reproducible and functional hydrogel can be produced. See, e.g., Salinas et al., Chondrogenic Differentiation Potential of Human Mesenchymal Stem Cells Photo encapsulated within Poly(Ethylene Glycol)-Arginine-Glycine-Aspartic Acid-Serine Thiol-Methacrylate Mixed-Mode Networks, Tissue Engineering 13 (5): 1025-34 (2007). In some embodiments, the hydrogel comprises a naturally-derived hydrogel such as alginate in combination with synthetic hydrogel, such as PEG or PVA. Alginate as used herein includes the native form of alginate as well as modified forms that are rendered biodegradable in vivo. See, e.g., US 2002/0168406 to Goldenberg et al.

In particular embodiments, the hydrogel is a mixture of alginate and gelatin (e.g., about 1%, 2% or 3% by weight of an alginate and gelatin mixture). In some embodiments, the alginate and gelatin are provided at a ratio of from 2:1 or 1.5:1, to 1:1.5 or 1:2 by weight of alginate:gelatin (e.g., about a 1:1 ratio).

In some embodiments, the hydrogel may include chemical or ionic crosslinking agents. Non-limiting examples include calcium carbonate (CaCO₃) and glucono-δ-lactone (GDL). See, e.g., US Patent Application Publication No. 2019/0284349 to Bassett et al.

In some embodiments, the carrier may be a cream or gel suitable for topical application to a would such as a skin wound.

In some embodiments, the carrier is in the form of a patch or sheet. For example, the carrier may include synthetic aliphatic polyesters such as poly(glycolic acid) (PGA), poly(L-lactic acid) (PLA), or poly(lactic-co-glycolic acid) (PLGA), and/or natural polymers such as chitosan, keratin, or silk fibroin. In some embodiments, the carrier may be formed by electrospinning. See, e.g., U.S. Pat. No. 8,728,498 to Zhang et al.

In some embodiments, the carrier comprising active agents may be frozen for storage, and can be thawed (and optionally warmed to room temperature or body temperature) shortly before use. In some embodiments, the carrier (e.g., a hydrogel carrier) may be freeze-dried (which may form a foam). In some embodiments, a foam carrier is preferred. In some embodiments, the carrier may include a softening agent such as glycerol.

In particular embodiments, the foam comprises a mixture of alginate and gelatin. In some embodiments, the alginate and gelatin are provided in the foam at a ratio of from 2:1 or 1.5:1, to 1:1.5 or 1:2 by weight of alginate:gelatin (e.g., about a 1:1 ratio).

In addition to the active agents, the compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include sodium benzoate, potassium sorbate, methyl hydroxybenzoate (methylparaben), EDTA, sorbic acid, parabens, thimerosal, etc.

Useful microbial preservatives may also include methylparaben, propylparaben, and benzyl alcohol. Dyes or other coloring agents may be included, as well. Protein stabilizers, such as sugars (e.g., sucrose, glucose, trehalose) and polyols (e.g., polyethylene glycol), may also be included.

### II. Methods of Use

Compositions as taught herein may be used for treating a subject in need thereof, such as a method for treating a wound (e.g., a skin wound), with application of the composition to said wound. The composition may be applied in an amount effective to accelerate healing of the wound (e.g., accelerate wound closure, tissue formation, and/or re-epithelialization), and/or in an amount effective to accelerate angiogenesis of the wound. In some embodiments, the composition is applied to said wound in an amount effective to reduce scarring (e.g., by promoting basket-weave collage network formation during healing).

In some embodiments, the wound is hypoxic. Hypoxic wounds do not have adequate oxygen/oxygenation to support healing, and thus cannot heal properly. In some embodiments, the wound is a burn wound. In some embodiments, the wound is an acute skin wound. In some embodiments, the wound is a full-thickness skin wound such as a full-thickness acute skin wound.

In some embodiments, the wound is a chronic skin wound. In some embodiments, the wound is a full-thickness chronic skin wound (e.g., a chronic ulcer).

Compositions as taught herein may also be used to promote cell proliferation in vitro or in vivo. For example, cells may be cultured in vitro in the presence of a composition as taught herein, in which the factors may be provided in an amount effective to promote proliferation of the cells. For cell delivery in vivo, a composition as taught herein which contains said cells may be delivered to a subject in need thereof.

The present invention is explained in greater detail in the following non-limiting Examples.

### Examples

### Example 1: Identification and Development of Conditioned Medium Factors

This study developed a strategy to identify stem cell secretome-based recombinant proteins (referred to as conditioned medium factors (CMF)) for wound healing applications. The specific protein combination determined by the stem cell-derived secretome analysis could be developed as a therapy, as a form of recombinant protein cocktail. This strategy of using identified recombinant proteins has many advantages over using conditioned media (CM). Using a defined composition and dosage of the recombinant protein cocktail (CMF) eliminates the need for extensive cell culture and CM collection. It would maintain a therapeutically consistent product, thereby providing an off-the-shelf option that could be useful for the acute phase of the wound and burn care.

We analyzed the secretome profile of hPSC-derived CM-cultivated in normoxia and hypoxia by proteomics and identified the proteins that are effective in wound healing and regeneration. Several studies reported that stem cells secrete antiapoptotic, pro-proliferative, and pro-angiogenic factors under hypoxic conditions (Das et al., 2010 Tissue Eng. Part B Rev. 16 (2), 159-168; Chen et al., 2014 PLoS One 9 (4), e96161; Jun et al., 2014 Int. J. Mol. Sci. 15 (1), 605-628). The hPSC secretome profile in hypoxia can be different from that in normoxia. Given that wound beds are typically hypoxic, it was necessary to investigate the secretome profile of hPSC-derived CM in the hypoxic environment. We developed a stem cell secretome-based recombinant protein cocktail (CMF) that synergistically affects the regenerative capacities of the human dermal fibroblasts (HDF) and endothelial cells responsible for wound healing. We hypothesized that the CMF treatment would enhance HDF growth and migration and prevent apoptotic cell death, which are the characteristics of cellular response in wound healing. We studied the concentration of CMF in terms of HDF growth.

To determine the effectiveness of the protein combination, HDF growth under protein cocktail treatment was compared with those of each protein treatment. The treatment efficacy of CMF on HDF proliferation, anti-apoptosis, and migration was evaluated and compared with hPSC-derived CM treatment. In addition, the CMF treatment effects on promoting angiogenesis were assessed using an in ovo chick chorioallantoic membrane (CAM) assay. The effect of CMF on endothelial cell growth was also evaluated.

### Materials and Methods

### Cell Culture

hPSCs isolated from donated human placenta were obtained from the Manufacturing Development Center at Wake Forest Institute for Regenerative Medicine (Zhang et al., 2016; Yim et al., 2019). Human placental stem cells (hPSCs) (passage 6-7) were cultured in hPSC growth medium (hPSC-GM) containing 65% of Alpha Minimum Essential Medium (α-MEM; Gibco, Amarillo, TX), 17% Amniomax C100 Basal Medium (Gibco), 15% Fetal Bovine Serum (FBS; Gibco), 2% Aminomax Supplement (Gibco), 1% Glutamax (Gibco), and 2.5 μg/ml Gentamicin (Gibco). HDFs isolated from donated infant foreskin were obtained from the Wake Forest Institute for Regenerative Medicine Skin Team. HDFs were cultured in Dulbecco's Modified Eagle Medium (DMEM; Cytiva; Logan,UT, United States) containing 10% FBS and 1% Penicillin/Streptomycin (P/S; Fischer Scientific, Waltham, MA) (HDF-GM) (passage 5-7) (Jorgensen et al., 2020 Tissue Eng. Part A 26 (9-10), 512-526). The medium was changed every 2-3 days. Human umbilical vein endothelial cells (HUVECs) (Lonza, Basel, Switzerland) were cultured in HUVEC growth medium (HUVEC-GM) containing Endothelial Cell Basal Medium-2 (EBM-2; Lonza) and EBM-2 SingleQuots (EBM-2-SQ; 2% Fetal Bovine Serum, 0.04% Hydrocortisone, 0.4% hFGF-B, 0.1% VEGF, 0.1% R3-IGF-1, 0.1% Ascorbic Acid, 0.1% hEGF, 0.1% GA-1000, 0.1% Heparin; Lonza) (passage 3). The medium was changed every 2-3 days.

Preparation of hPSC-Derived Conditioned Medium and Proteomics Analysis

hPSCs (10,000 cells/cm²) were plated in a 15 cm culture dish and cultured overnight in hPSC-GM. The cells were re-fed with serum-free hPSC medium (hPSC-SFM; hPSC-GM without FBS and Amniomax Supplement) and incubated in normoxia (21% O₂) and hypoxia (1% O₂) (Hypoxia C-chamber, Biospherix, Parish, NY, United States) conditions for 3 days, respectively. hPSC-CM obtained from normoxia (hPSC-CM-N) and hypoxia (hPSC-CM-H) were collected and centrifuged at 1500 rpm for 5 min. The supernatant was collected to eliminate cellular debris. The hPSC-CM was concentrated 4-fold (4×) using Amnicon Ultra-15 Centrifugal Filter Units with a molecular cutoff of 3 kDa (Millipore Sigma, Germany). The concentrated hPSC-CM was frozen and stored at −80° C. until use. For proteomics, hPSC-CM was collected from 3 different donors. The 4-fold (4×) concentrated hPSC-CM was analyzed for 200 human protein content using Quantibody Human Cytokine Array Q4000 (Quantibody quantitative multiplex ELISA array, catalog #QAH-CAA-4000: Raybiotech, Norcross, GA).

### Conditioned Medium Factor Concentration Test

Nine proteins effective for wound healing were selected based on the normoxic hPSC-CM (hPSC-CM-N) and hypoxic hPSC-CM (hPSC-CM-H) analysis. The proteins that promoted wound healing and angiogenesis and were expressed at higher-than-normal serum levels in hPSC-CM-N and hPSC-CM-H were selected. The proteins possessing inhibitory effects were excluded from the selection. The combination of the nine proteins was prepared using recombinant human proteins (CMF). The CMF includes 1) ANG-1 (R&D Systems, Minneapolis, MN), 2) FGF-7 (R&D Systems), 3) Follistatin (Sigma-Aldrich, St. Louis, MO), 4) HGF (Sigma Aldrich), 5) Interleukin-6 (IL-6; R&D Systems), 6) Insulin (Sigma Aldrich), 7) Transforming growth factor beta-1 (TGFβ-1; R&D Systems), 8) Urokinase receptor (uPAR; R&D Systems), and 9) VEGF (R&D Systems) (Table 1). The concentration of ANG-1, FGF-7, Follistatin, HGF, IL-6, TGFβ-1, and uPAR was determined to be equivalent to the concentration in hPSC-CM-N, because the protein concentration in hPSC-CM-N and hPSC-CM-H was not statistically different (p>0.05). The Insulin and VEGF concentration was determined to be equivalent to the concentration in hPSC-CM-H, because these protein concentrations were higher in hPSC-CM-H.

TABLE 1
Nine proteins highly expressed in normoxia and hypoxia with major roles in wound healing are identified.

| | | | | Recombinant | |
|---|---|---|---|---|---|
| | Normoxia | Hypoxia (4x, | Major Role in Wound | proteins (CMF 4x, | |
| Protein | (4x, ng/mL) | ng/mL) | Healing | ng/mL) | Reference |
| ANG-1 | 4.223 ± 1.950 | 2.367 ± 0.332 | Angiogenesis | 4.223 | 1, 2, 3, 4 |
| FGF-7 | 1.437 ± 0.637 | 0.222 ± 0.205* | Re-epithelialization | 1.437 | 1, 5, 2, 6 |
| Follistatin | 11.052 ± 4.274 | 9.496 ± 2.422 | Regulate fibrosis and | 11.052 | 7, 8, 9 |
| | | | reduce scar formation | | |
| HGF | 16.670 ± 0.582** | 14.895 ± 0.636** | Angiogenesis | 16.670 | 1, 2, 3, 10, |
| | | | Re-epithelialization | | 6 |
| | | | Endothelial cell and | | |
| | | | fibroblast | | |
| | | | proliferation and | | |
| | | | migration | | |
| IL-6 | 5.372 ± 1.562 | 6.017 ± 0.648** | Wound closure, Re- | 5.372 | 1, 3, 11 |
| | | | epithelialization, | | |
| | | | collagen production | | |
| | | | Fibroblast migration | | |
| | | | and differentiation | | |
| | | | Angiogenesis | | |
| Insulin | 6.293 ± 2.193 | 16.763 ± 5.635 | Angiogenesis | 16.763 | 12, 13 |
| TGFβ-1 | 41.487 ± 35.207 | 14.935 ± 3.295 | Collagen production, | 41.487 | 14, 5, 15, |
| | | | Wound closure and | | 16, 4 |
| | | | contraction | | |
| | | | Fibroblast | | |
| | | | proliferation, | | |
| | | | migration and | | |
| | | | differentiation | | |
| | | | Angiogenesis | | |
| uPAR | 5.223 ± 0.692 | 7.301 ± 1.859 | Extracellular matrix | 5.223 | 17, 18, 19, |
| | | | remodeling | | 20, 21, 22, |
| | | | Fibroblast and | | 23 |
| | | | endothelial cells | | |
| | | | migration | | |
| | | | Angiogenesis | | |
| VEGF | 0.0 ± 0.0* | 2.303 ± 0.762 | Angiogenesis | 2.303 | 1, 2, 3, 4, 6 |

*LOD (value below the limit of detection),
**MAX (value above the highest standards). ANG-1: Angiopoietin-1; FGF-7: Fibroblast growth factor-7; HGF: Hepatocyte growth factor; IL-6: Interleukin-6; TGFβ-1: Transforming growth factor beta-1; uPAR: Urokinase receptor; VEGF: Vascular endothelial growth factor. References: 1. Warner and Grose, 2003; 2. Ayman Grada, 2018; 3. Ferreira and Gomes, 2018; 4. Ahangar et al., 2020; 5. Barrientos et al., 2008; 6. Huang et al., 2020; 7. McDowall et al., 2008; 8. de Kretser et al., 2012; 9. Ham et al., 2021; 10. Park et al., 2018; 11. Johnson et al., 2020; 12. Liu et al., 2009; 13. Escudero et al., 2017; 14. Clark et al., 1997; 15. Pakyari et al., 2013; 16. Liu et al., 2016; 17. Solberg et al., 2001; 18. Blasi and Cameliet, 2002; 19. Li et al., 2003; 20. D'Alessio et al., 2008; 21. Rao et al., 2013; 22. Cooper et al., 2018; 23. Mahmood et al., 2018.

To determine the optimal CMF concentration, various concentrations of CMF were tested using HDFs. HDFs (5000 cells/well) were plated in 48 well plates in HDF-GM overnight. HDFs were treated with HDF-serum free medium (HDF-SFM; DMEM and 1% P/S), CMF 1×, CMF 2×, CMF 4×, CMF 6×, CMF 8×, and CMF 10× and cultured under normoxia and hypoxia, respectively (Table 2). The concentration of each protein in the CMF 4× was equivalent to that in the 4× concentrated CM obtained from the hPSC-CM analysis (Table 1). The concentrations of each protein in all other CMF groups were calculated from the analyzed CM protein concentration.

TABLE 2
Various CMF concentrations (nine protein combination)
tested on human dermal fibroblasts (CMF 1×,
2×, 4×, 6×, 8×, and 10×).

| Protein | | | | | | |
|---|---|---|---|---|---|---|
| (ng/mL) | CMF 1x | CMF 2x | CMF 4x | CMF 6x | CMF 8x | CMF 10x |
| ANG-1 | 1.056 | 2.112 | 4.223 | 6.335 | 8.446 | 10.558 |
| FGF-7 | 0.359 | 0.719 | 1.437 | 2.156 | 2.874 | 3.593 |
| Follistatin | 2.763 | 5.526 | 11.052 | 16.578 | 22.104 | 27.630 |
| HGF | 4.168 | 8.335 | 16.670 | 25.005 | 33.340 | 41.675 |
| IL-6 | 1.343 | 2.686 | 5.372 | 8.058 | 10.744 | 13.430 |
| Insulin | 4.191 | 8.382 | 16.763 | 25.145 | 33.526 | 41.908 |
| TGFβ-1 | 10.372 | 20.744 | 41.487 | 62.231 | 82.974 | 103.718 |
| uPAR | 1.306 | 2.612 | 5.223 | 7.835 | 10.446 | 13.058 |
| VEGF | 0.576 | 1.152 | 2.303 | 3.455 | 4.606 | 5.758 |

After treatment for 0, 2, and 5 days, the alamarBlue assay was performed according to the manufacturer's instruction (Invitrogen; Waltham, MA). Briefly, the cells were incubated in alamarBlue assay reagent (1:10 dilution) for 2 h at 37° C. The incubated reagent was transferred to clear flat bottom 96 well plates, and fluorescence was measured at a wavelength of 560 and 590 nm (excitation and emission) (Spectra Max MS multiplate reader, Molecular Devices, San Jose, CA, United States) (n=3 per group and time point, duplicate measure). The measured optical density (OD) value was converted to cell number using a standard curve. To develop the standard curve between OD value and known HDF cell number, HDFs were seeded at a density of 0, 1563, 3125, 6250, 12,500, 25,000, 50,000, and 100,000 cells/well and cultured overnight.

The OD Value Corresponding to the Cell Number was Obtained Using the alamarBlue Assay (n=3).

Comparison of cell growth between single protein and combinatorial proteins The effectiveness of the recombinant protein combination (CMF) compared with individual proteins on cell growth was evaluated using an alamarBlue assay. HDFs (5000 cells/well) were cultured overnight in HDF-GM in 48 well plates. The cells were treated with HDF-GM, HDF-SFM, CMF composed of nine recombinant human protein cocktails (ANG-1, FGF-7, Follistatin, HGF, IL-6, Insulin, TGFβ-1, uPAR, VEGF), or individual proteins. In this study, CMF 6×was tested because the CMF 6×was determined as the optimal CMF concentration in the above CMF concentration study. The concentration of individual protein was equivalent to that in the CMF 6×. After the treatment, the cells were cultured in normoxia and hypoxia, respectively. On days 0, 2, and 5, the alamarBlue assay was performed (n=3 per group and time point, duplicate measure). The measured OD value was converted to cell numbers using a standard curve described above. The cell number of each group was normalized to the day 0 cell number.

### Effects of Condition Medium Factors on Human Dermal Fibroblast Growth

The effect of CMF on HDF growth was compared with hPSC-CM and co-culture with hPSC. HDFs (10,000 cells/well) were plated in 24 well plates overnight in HDF-GM and then treated with HDF-GM, HDF-SFM, hPSC-CM-N 6× or hPSCCM-H 6×, and CMF 6×. For the co-culture group, hPSC (0.315×106 cells) were plated in the transwell insert (Transwell Permeable Supports; Corning, NY) in hPSC-GM. hPSC seeding density used for the co-culture and CM 6× collection was the same. Following overnight culture, the hPSC-seeded insert was combined with the HDF-seeded 24 well plates and cultured in HDF-SFM. For the hPSC-CM group, the CM was concentrated 10-fold (10×) using Amnicon Ultra-15 Centrifugal Filter Units and diluted to 6×concentration with HDF-SFM. Test samples were treated with CM collected under normoxia (hPSCCM-N) and CM collected under hypoxia (hPSC-CM-H), in normoxia and hypoxia, respectively. The CM samples were pooled from the donors that were tested in the proteomics study. All test samples were cultured in normoxia and hypoxia, and an alamarBlue assay was performed on days 0, 2, and 5. The OD value of each sample was converted to the cell number using a standard curve. The cell number of each group and time point was normalized to the cell number at day 0. The standard curve was developed with the seeding density of 0, 3125, 6250, 12,500, 25,000, 50,000, 100,000, and 200,00 cells/well in 24 well plates (n=3 per group and time point, duplicate measure).

### Proliferating Nuclear Cell Antigen and Cleaved Caspase-3 ELISA Assay

Apoptosis and proliferation of HDFs were evaluated with Cleaved Caspase-3 (CC3) ELISA Kit (Abcam, Cambridge, MA, United States) and Proliferating Nuclear Cell Antigen (PCNA) ELISA Kit (Abcam) (n=3). HDFs (0.75×106 cells/10 cm dish) were cultured overnight in HDF-GM and then treated with HDF-GM, HDF-SFM, hPSC-CM-N 6× or hPSC-CM-H 6×, and CMF 6× for 3 days in normoxia and hypoxia, respectively. HDF-SFM was used to dilute hPSC-CM 10× stock to hPSC-CM 6×. The treatment medium was decanted, and HDFs were lysed by incubating the HDFs in 1×RIPA buffer (Cell Signaling Technology, Danvers, MA) with a protease inhibitor cocktail (1:100; Cell Signaling Technology) for 15 min on ice. Lysed cells were centrifuged at 18,000 g for 20 min at 4° C., and the supernatant was collected. The total protein concentration of cell lysed supernatant was determined using BCA Protein Assay Kit (Thermo Scientific, Rockford, IL, United States) according to the manufacturer's instruction. For PCNA and CC3 ELISA, the cell lysate was adjusted to 5 μg/ml and 1000 μg/ml, respectively. ELISA assay was performed according to the manufacturer's instructions.

### In Vitro Wound Healing Assay

CytoSelect Healing Assay (Cell Biolabs, San Diego, CA, United States) evaluated wound healing in vitro. HDFs (0.75×10⁵ per/well) were seeded in a 24-well plate with CytoSelect wound healing inserts and cultured overnight in HDF-GM to form a monolayer around the insert. The insert was removed, leaving an approximate 0.9 mm wound field between cells. The cells were treated with HDF-GM, HDFSFM, hPSC-CM-N 6× or hPSC-CM-H 6×, and CMF 6× and cultured for 1 day in normoxia and hypoxia, respectively. hPSC CM 6× was diluted from hPSC-CM 10× stock using HDF-SFM. Cytoselect cell stain solution was added to each well for 15 min, washed 3 times with deionized water, and imaged using a light microscope (Olympus, Tokyo, Japan). The percentage of cell migration to the wound field was measured using Image J software (National Institutes of Health, Bethesda, MD) (n=4 per group, 6 random fields per sample).

### In Ovo Chorioallantoic Membrane Assay

To investigate the angiogenic effect of CMF, CAM Assay was performed in ovo (n=6). Fertilized eggs were obtained from SNL Feeds (Lexington, NC, United States) and developed in the Intelligent Egg Incubator 56S (Ridgeyard, China) at 37° C. for 72 h at a constant humidity of 50%. On day 3 of development, 4 ml of albumin was removed from each egg using a sterile 25-gauge needle, and a circular window (1×1 cm2) was cut into the top of each eggshell. The window was sealed with Tegaderm film (3M Science, Minneapolis, MN, United States), and the eggs were placed into the incubator for an additional 5 days (day 8 of development). Chick embryo and vessel development were observed each day. On day 8 of development (day 0 of CAM assay), CMF 6× (20 i) and the control (PBS, (20 μl) were mixed and absorbed into a gelatin-based scaffold (5 mm diameter) (Gelfoam©, Upjohn, Kalamazoo, MI, United States) respectively, and placed on top of the CAM. The CAM was imaged on day 0 using a stereoscopic zoom microscope (Nikon SMZ-U, Tokyo, JP), and vessels peripheral to the scaffold were counted using Image J software. On day 5 of the CAM assay (day 13 of development), CAM was imaged, and vessels peripheral to the scaffold were counted. The day 5 vessel count was subtracted from the day 0 vessel count to determine the number of newly formed vessels (n=6 per group and time point). Following imaging, day 5 gel foam constructs were harvested, fixed with 4% paraformaldehyde, processed and embedded with paraffin, sectioned at 5 μm, and placed on histology slides. Each sample was sectioned in a horizontal and vertical orientation, stained with hematoxylin and eosin (H&E), and imaged using a light microscope. Stained vessels of the horizontal sections were counted using Image J software (n=4 per group).

### Effects of Conditioned Medium Factors on Endothelial Cell Growth

The effect of CMF on HUVEC growth was compared with hPSC-derived CM and HUVEC/hPSC co-culture. The HUVEC (5000 cells/well, passage 3) was plated in 24 well plates overnight in HUVEC-GM and then treated with HUVEC serum free medium (HUVEC-SFM; EBM-2 with 0.1% (w/v) bovine serum albumin (BSA; MP Biomedicals, Solon, OH, United States)), hPSC-CM-N 6× for normoxia, hPSC-CM-H 6× for hypoxia, and CMF 6×, respectively. For the co-culture group, hPSC (0.315×10⁶ cells) were plated in transwell inserts (Transwell Permeable Supports; Corning, NY, United States) in hPSC-GM. For the hPSC-CM groups, HUVEC-SFM was used to dilute hPSC-CM 10× stock to hPSC-CM 6×. The hPSC-seeding density used for the co-culture and CM collection was the same. Following overnight culture, the hPSC-seeded insert was combined with the HUVEC-seeded 24 well plates and cultured in a HUVEC-SFM medium. The test samples were cultured in normoxia (21% 02) and hypoxia (1% 02). AlamarBlue assay was performed on days 0, 2, and 3. The OD value of each sample was converted to the cell number using a standard curve. The cell number of each group and time point was normalized to the cell number at day 0. The standard curve was developed with the seeding density of 0, 3125, 6250, 12,500, 12,500, 25,00, 50,000, 100,000, 200,000 cells/well in 24 well plates (n=4 per group and time point, duplicate measure).

### Statistical Analysis

Results were analyzed with Origin Pro 8.5 (OriginLab Co, Northampton, MA), GraphPad Prism 8 (GraphPad Software, San Diego, CA, United States), and SPSS software (SPSS, version 19; IBM, Armonk, NY, Unites States). T-test, one-way or two-way analysis of variance (ANOVA), and Tukey's post hoc tests were applied to mean comparisons. Variables are expressed as a mean±standard deviation (SD), and differences between experimental groups were considered statistically significant at p<0.05.

### Results

hPSC-Derived Conditioned Medium Analysis

The secretome profile was obtained by proteomic analysis of hPSC-CM obtained under normoxic and hypoxic conditions (hPSC-CM collected from 3 different donors, n=3 samples from each batch of hPSC-CM). Nine proteins that promote wound healing and angiogenesis and are expressed at higher-than-normal serum levels in hPSC-CM-N and hPSC-CM-H (Sakamoto et al., 1996 Eur. J. Endocrinol. 135 (3), 345-351; Kyrtsonis et al., 1998 Med. Oncol. 15 (2), 124-128; Miyake et al., 1999 Prostate 39 (2), 123-129; Di Raimondo et al., 2001 Leukemia 15 (6), 976-980; Malatino et al., 2003 Hypertension 41 (1), 88-92; Nockowski et al., 2004 Acta dermatovenerol. Croat. 12 (1), 2-6; Anargyrou et al., 2008 Haematologica 93 (3), 451-454; Albanes et al., 2009 J. Natl. Cancer Inst. 101 (18), 1272-1279; Jing et al., 2012 J. Clin. Lab. Anal. 26 (1), 16-21; Ren et al., 2012 J. Int. Med. Res. 40 (3), 877-886; Kleiner et al., 2013 Mediat. Inflamm. 2013, 1-6; Moon et al., 2014 BMC Pulm. Med. 14, 143; Noman et al., 2017 Sci. Rep. 7 (1), 1796; Kritmetapak et al., 2021 BMC Nephrol. 22 (1), 114; Said et al., 2021 J. Med. Virol. 93 (6), 3915-3924) were selected. Any proteins possessing inhibitory effects for wound healing were excluded from consideration in this study. The concentration and major roles of these nine proteins are reflected in Table 1. ANG-1, FGF-7, Follistatin, HGF, IL-6, TGFβ-1, and uPAR, were highly expressed in both normoxic and hypoxic hPSC-CM; the protein concentration in normoxic hPSC-CM and hypoxic hPSC-CM was not statistically different (p<0.05, Student t-test). Insulin was highly expressed in both normoxic and hypoxic hPSC-CM, but the concentration of Insulin was significantly higher in hypoxic hPSC-CM than in normoxic hPSC-CM (p<0.05, Student t-test). In addition, VEGF was highly expressed in hypoxic hPSC-CM but was not detected in normoxic hPSC-CM.

These nine proteins are associated with accelerating wound healing and/or angiogenesis. The major role of FGF-7 (Werner and Grose, 2003 Physiol. Rev. 83 (3), 835-870; Barrientos et al., 2008 Wound Repair Regen. 16 (5), 585-601; Grada and Falanga, 2018 “Stem cell therapies for wound healing,” in Chronic wounds, wound dressings and wound Healing, Recent clinical techniques, results, and research in wounds, 301; Huang et al., 2020 Tissue Eng. Part B Rev. 26 (6), 555-570), Follistatin (McDowall et al., 2008 Cytokine Growth Factor Rev. 19 (5-6), 415-426; de Kretser et al., 2012 Mol. Cell. Endocrinol. 359 (1-2), 101-106; Ham et al., 2021 Exp. Dermatol. 30 (3), 402-408), IL-6 (Werner and Grose, 2003 Physiol. Rev. 83 (3), 835-870; Ferreira and Gomes, 2018 Bioeng. (Basel) 6 (1), 4; Johnson et al., 2020 Biomedicines 8 (5), 101), TGFβ-1 (Clark et al., 1997 J. Cell Physiol. 170 (1), 69-80; Barrientos et al., 2008 Wound Repair Regen. 16 (5), 585-601; Pakyari et al., 2013 Adv. Wound Care (New. Rochelle) 2 (5), 215-224; Liu et al., 2016 Sci. Rep. 6, 32231; Ahangar et al., 2020 Int. J. Mol. Sci. 21 (19), 7038), and uPAR (Solberg et al., 2001 J. Histochem. Cytochem. 49 (2), 237-246; Blasi and Carmeliet, 2002 Nat. Rev. Mol. Cell Biol. 3 (12), 932-943; Li et al., 2003 Wound Repair Regen. 11 (4), 239-247; D'Alessio et al., 2008 J. Cell Sci. 121 (Pt 23), 3922-3932; Rao et al., 2013 Oncogenesis 2, e53; Cooper et al., 2018 J. Invest. Dermatol. 138 (11), 2470-2479; Mahmood et al., 2018 Front. Oncol. 8, 24) includes promoting re-epithelialization, extracellular matrix production and wound closure. ANG-1 (Werner and Grose, 2003 Physiol. Rev. 83 (3), 835-870; Ferreira and Gomes, 2018 Bioeng. (Basel) 6 (1), 4; Grada and Falanga, 2018 “Stem cell therapies for wound healing,” in Chronic wounds, wound dressings and wound Healing, Recent clinical techniques, results, and research in wounds, 301; Ahangar et al., 2020 Int. J. Mol. Sci. 21 (19), 7038), HGF (Werner and Grose, 2003 Physiol. Rev. 83 (3), 835-870; Ferreira and Gomes, 2018 Bioeng. (Basel) 6 (1), 4; Grada and Falanga, 2018 “Stem cell therapies for wound healing,” in Chronic wounds, wound dressings and wound Healing, Recent clinical techniques, results, and research in wounds, 301; Park et al., 2018 Mol. Ther. 26 (2), 606-617; Huang et al., 2020 Tissue Eng. Part B Rev. 26 (6), 555-570), Insulin (Liu et al., 2009 J. Cell. Mol. Med. 13 (11-12), 4492-4504; Escudero et al., 2017 Front. Physiol. 8, 204), and VEGF (Werner and Grose, 2003 Physiol. Rev. 83 (3), 835-870; Ferreira and Gomes, 2018 Bioeng. (Basel) 6 (1), 4; Grada and Falanga, 2018, supra; Ahangar et al., 2020, supra; Huang et al., 2020, supra) promote angiogenesis.

Based on the proteomics study, the composition and concentration of nine recombinant proteins as a wound healing therapy (CMF) were determined. The normoxic hPSC-CM concentrations in Table 1 for all recombinant proteins except Insulin and VEGF were selected for use in this study. As Insulin and VEGF concentrations were higher in hypoxic hPSC-CM, the recombinant Insulin and VEGF concentrations selected were the same concentration as in hypoxic hPSC-CM (Table 1).

### Conditioned Medium Factor Concentration

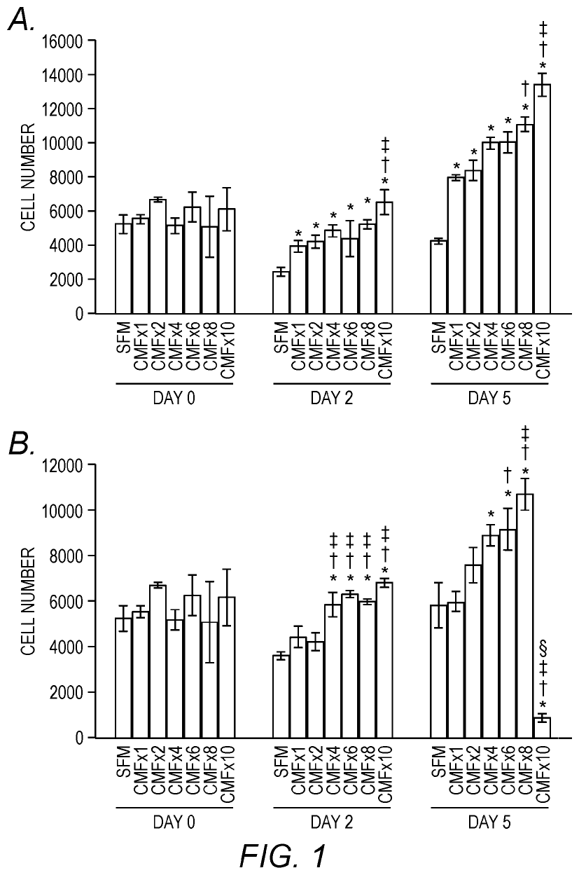

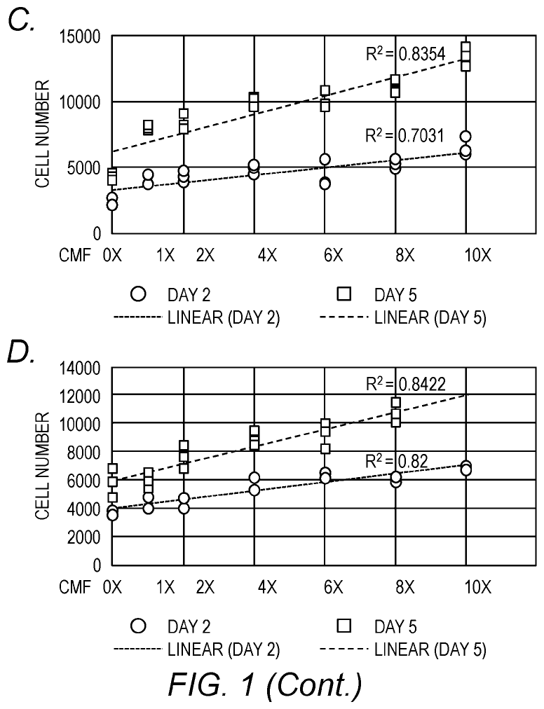

We investigated the effect of CMF concentration on HDF growth to determine the optimal CMF concentration. HDF was cultured in various concentrations of CMF in normoxia and hypoxia, respectively (Table 2), and cell number was calculated on days 0, 2, and 5 (n=3, two-way ANOVA and Tukey's test) (FIG. 1 Panels A,B). No significant difference was detected in the HDF number at day 0 in normoxia and hypoxia (p>0.05). In normoxia, all the CMF treatment groups (CMF 1×, 2×, 4×, 6×, 8×, and 10×) showed significantly increased cell numbers compared with SFM(CMF 0×) on days 2 and 5 (*p<0.05) (FIG. 1, Panel A). HDF number of CMF 8× was significantly increased compared with CMF 1× (tp<0.05) on day 5. HDF number of CMF 10× was higher than CMF 1× (†p<0.05), 2×, 4×, and 6× on day 2 and day 5 (‡p<0.05). In hypoxia, CMF 4×6×, 8×, and 10× showed significantly increased HDF number compared with SFM (*p<0.05), CMF 1×, and 2× (†p<0.05) on day 2; however, there was no significant difference between CMF 4×, 6×, 8×, and 10× (p>0.05) (FIG. 1, Panel B). On day 5 in hypoxia, significantly increased cell number was detected in CMF 6× and 8×treatments in comparison to SFM (*p<0.05) and CMF 1× (†p<0.05) (FIG. 1, Panel B). CMF 8× showed a higher cell number than CMF 2× (*p<0.05). However, no significant difference was found between CMF 6× and 8× (p>0.05). Unexpectedly, the HDF number of CMF 10× decreased compared with the other groups on day 5 in hypoxia, indicating possible inhibitory effects of CMF 10× treatment in hypoxia.

To investigate further the relationship between CMF concentration and cell growth, linear regression analysis was performed. The CMF 10× concentration in hypoxia was excluded in this analysis due to the inhibitory effect on cell growth observed above. Linear regression indicates a strong positive relationship between CMF concentration and cell number, showing R²>0.7031 both in normoxia and hypoxia (FIG. 1, Panels C,D). These results indicate that CMF increases cell growth in a dose-dependent manner but not in concentrations greater than 6× (p>0.05 between CMF 6× and CMF 8×) (FIG. 1, Panel B). Overall, CMF at a concentration of 6× is the optimal concentration to enhance HDF growth and was selected and denoted as CMF throughout the remaining studies.

Effects of Recombinant Protein Combination (Conditioned Medium Factors) Compared with Individual Proteins

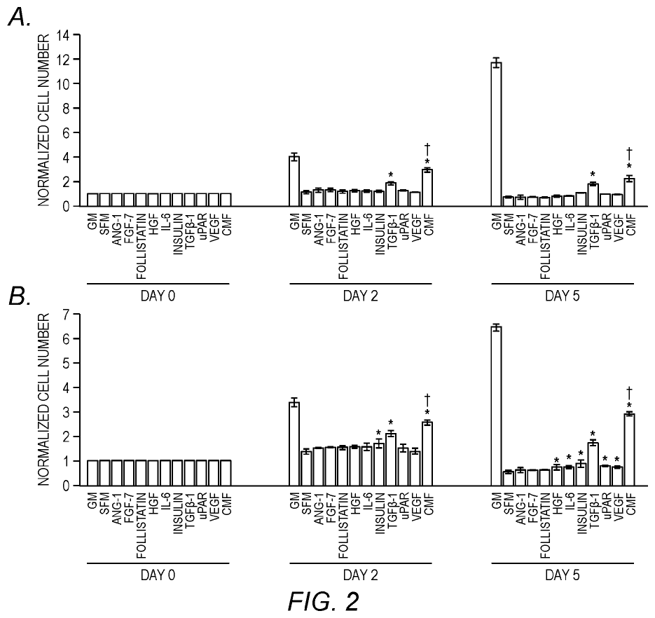

We compared the effect of recombinant protein combination CMF (CMF 6×) (ANG-1, FGF-7, Follistatin, HGF, IL-6, Insulin, TGFβ-1, uPAR, and VEGF) and individual proteins on HDF growth (n=3, two-way ANOVA and Tukey's test) (FIG. 2). On day 0 in normoxia and hypoxia, no significant difference was detected in HDF proliferation (p>0.05). On day 2 and day 5, in normoxia and hypoxia, TGFβ-1 and CMF showed significantly increased cell numbers compared with SFM (*p<0.05). The CMF group showed significantly increased cell numbers compared with all individual proteins (tp<0.05) on days 2 and 5 in hypoxia and normoxia conditions. On day 5, the HDF number of the CMF group increased 3-fold in normoxia and 5-fold in hypoxia compared with SFM. These results indicate that treatment with CMF yields higher cell growth than individual proteins.

Effects of CMF Compared with Conditioned Medium and hPSC Co-Culture

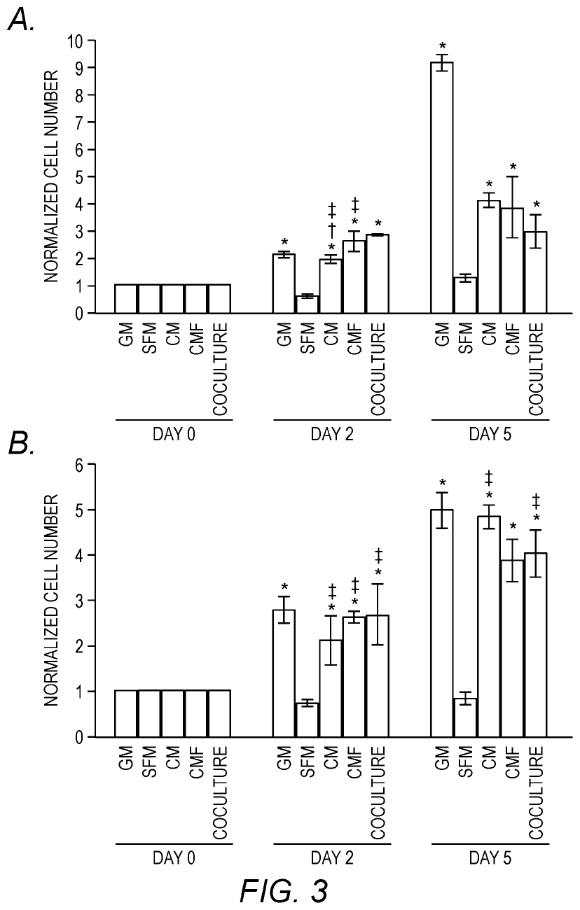

The effect of CMF, CM, and hPSC/HDF co-culture on HDF growth was investigated. HDF was cultured in normoxia and hypoxia with HDF-GM, SFM, hPSC-CM 6×, CMF 6×, and hPSC-HDF co-culture (n=3, two-way ANOVA and Tukey's test) (FIG. 3). HDF growth rates increased significantly with the treatment of CM, CMF, and co-culture compared to SFM on days 2 and 5 in normoxia and hypoxia (*p<0.05). CMF increased HDF number 3-fold and 5-fold compared to SFM at day 5 in normoxia and hypoxia (FIG. 3, Panels A,B), respectively. No significant difference was detected between CM, CMF, and coculture treatment groups on day 2 in hypoxia and day 5 in normoxia and hypoxia (p>0.05). These results indicate that CMF has a similar effect to CM and co-culture treatment on HDF growth.

### Human Dermal Fibroblasts Proliferation and Apoptosis by CMF Treatment

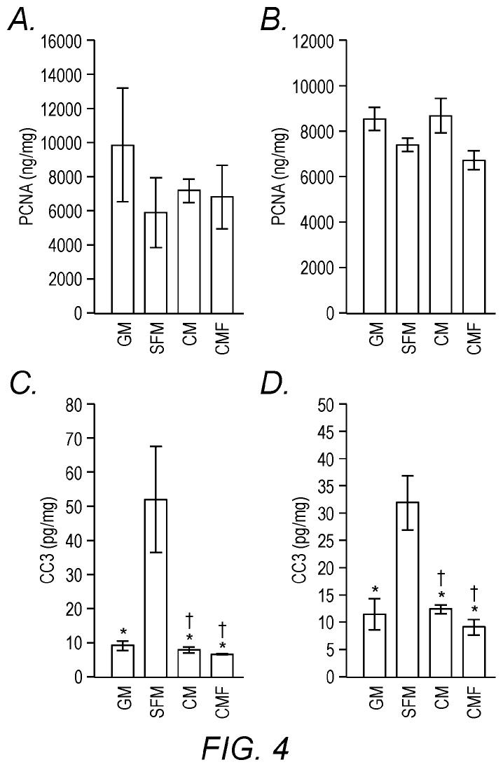

The pro-proliferative and anti-apoptotic effects of CMF were investigated using HDF. PCNA ELISA assay was performed to evaluate proliferation (n=3, one-way ANOVA and Tukey's test) (FIG. 4, Panels A,B). No significant difference was detected in PCNA protein levels between SFM, CM, and CMF groups in normoxia and hypoxia (p>0.05). CC3 ELISA assay was performed to evaluate apoptosis (n=3, one-way ANOVA and Tukey's test) (FIG. 4, Panels C,D). CC3 protein levels in HDF treated with CM and CMF were significantly decreased compared with SFM in normoxia and hypoxia (*p<0.05) and not statistically different compared with GM (†p>0.05). CC3 level with CMF treatment was decreased 3-fold in normoxia and 7-fold in hypoxia. CM and CMF showed no statistical difference in HDF apoptosis (p>0.05). These results indicate that CMF and CM prevent apoptosis but do not improve proliferation significantly.

### Human Dermal Fibroblasts Migration and Wound Closure by CMF Treatment In Vitro

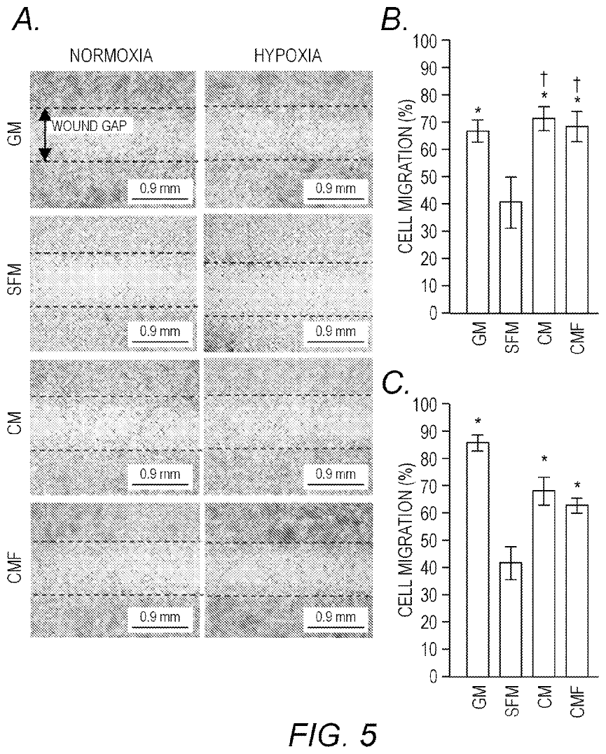

The effect of CMF on cell migration and wound closure was evaluated in vitro using CytoSelect Wound Healing Assay (n=4 per group, 6 random fields per sample, one-way ANOVA, and Tukey's test) (FIG. 5, Panels A,B). Cell migration (%) of GM, CM, and CMF increased significantly, approximately 2-fold, compared with SFM in normoxia and hypoxia (*p<0.05). There was no statistically significant wound closure effect between CM and CMF (p>0.05). These results indicate that CMF and CM positively affect HDF migration and wound closure with no statistical difference.

### Promoting Angiogenesis by Conditioned Medium Factor Treatment

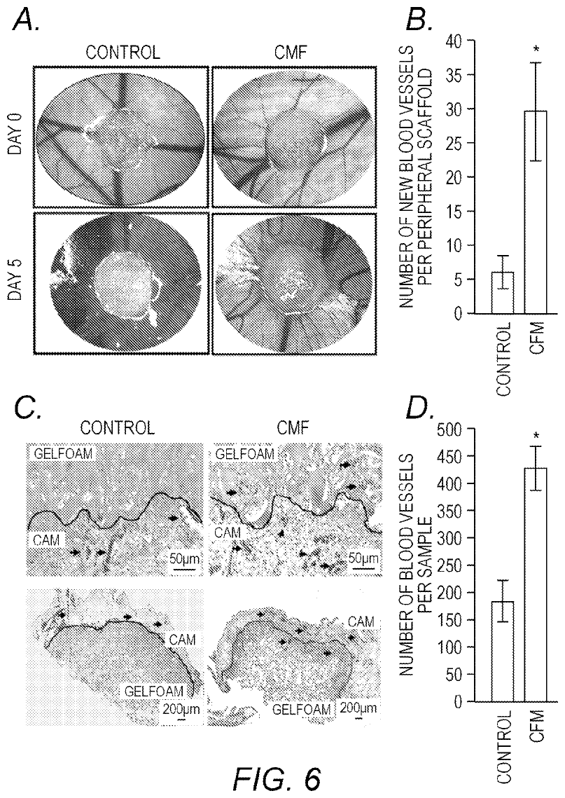

The angiogenic effect of CMF was investigated in ovo using chick CAM assay (n=6 per group, T-test, and Welch's correction). Gelfoam® containing CMF and the PBS (control) on CAM was imaged on days 0 and 5 after implantation. FIG. 6, Panel A shows the newly formed blood vessel number of the CMF group increased significantly, approximately 5-fold, compared with the control group (*p<0.05) (FIG. 6, Panel B). These results indicate that treatment with CMF yields a higher blood vessel number than the control group.

On day 5, Gelfoam® constructs with surrounding CAM were harvested and processed histologically. H&E staining of the vertical and horizontal sections shows vessel formation in the CAM and Gelfoam® area of the CMF group but not in the Gelfoam® area of the control treatment group (FIG. 6, Panel C—arrows). Blood vessel number per H&E sample of the CMF group increased significantly, approximately 2-fold, compared with the control (FIG. 6, Panel D) (n=4 per group, T-test, and Welch's correction, * p<0.05). These results indicate that treatment with CMF stimulates neovascularization and blood vessel formation in the scaffold compared to the control group.

### Human Umbilical Vein Endothelial Cell Growth by CMF Treatment

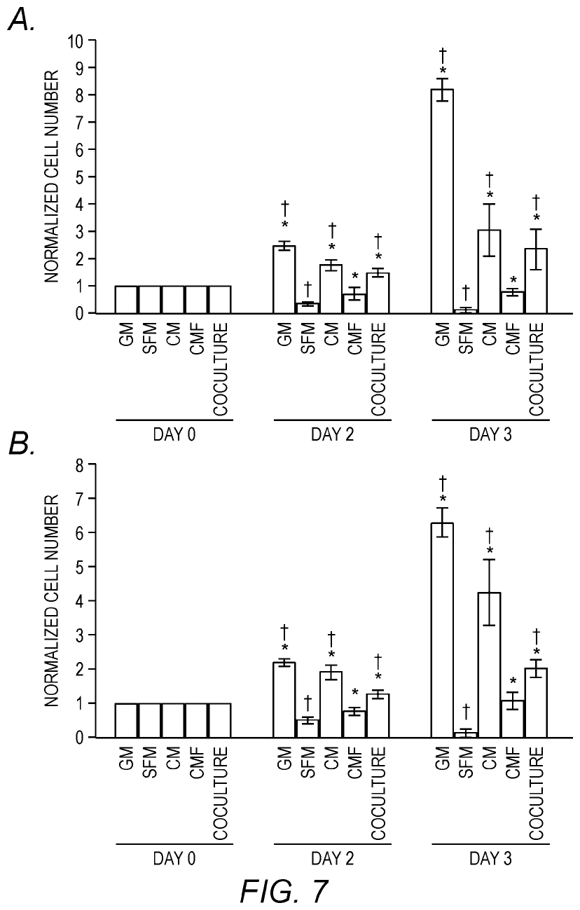

The effect of CMF, CM, and hPSC/HUVEC co-culture on HUVEC growth was investigated. HUVEC was cultured in normoxia and hypoxia with HUVEC-GM, HUVEC-SFM, hPSC-CM (6×), CMF (6×), and hPSC-HUVEC co-culture (n=3, two-way ANOVA and Tukey's test) (FIG. 7). No significant difference was detected in the HUVEC number at day 0 in normoxia and hypoxia (p>0.05). HUVEC growth increased significantly with the treatment of CMF, CM, and cocultured compared to SFM on days 2 and 3 in normoxia and hypoxia (*p<0.05) (FIG. 7). CMF increased HUVEC number 6-fold compared to SFM on day 3 in normoxia and hypoxia. CM increased HUVEC number 42-fold and 24-fold compared to SFM at day 3 in normoxia and hypoxia, respectively. Co-culture increased HUVEC number 17-fold and 11-fold compared to SFM at day 3 in normoxia and hypoxia, respectively. These results indicate that CMF significantly increases HUVEC growth but is less effective than the CM and co-culture groups.

### DISCUSSION

Stem cell-derived secretomes have been proposed as an alternate treatment modality to stem cell therapy for wound healing applications. However, stem cell-derived secretomes as a treatment modality is challenged by the safety concerns due to the undefined biomolecules in conditioned medium (CM). To that end, we developed an effective strategy to identify the prominent factors in CM that contribute to tissue regeneration and wound healing. In this study, nine defined recombinant proteins were selected to develop as a therapeutic cocktail (CMF) that promotes cell growth, accelerates cell migration and wound closure, prevents apoptosis in human dermal cells, and enhances angiogenesis associated with wound healing.

In this study, we performed a proteomic analysis of hPSC-derived CM to investigate major secretomes associated with wound healing and skin regeneration. We analyzed 200 human proteins in hPSC-derived CM cultured under normoxia and hypoxia. The proteomics results showed that the effective concentrations of ANG-1, FGF-7, Follistatin, HGF, IL-6, TGFβ-1, and uPAR were produced from hPSCs in both normoxic CM and hypoxic CM. These proteins play an important role in enhancing reepithelization, angiogenesis, and wound closure (Clark et al., 1997 J. Cell Physiol. 170 (1), 69-80; Solberg et al., 2001 J. Histochem. Cytochem. 49(2), 237-246; Blasi and Carmeliet, 2002 Nat. Rev. Mol. Cell Biol. 3 (12), 932-943; Li et al., 2003 Wound Repair Regen. 11 (4), 239-247; Werner and Grose, 2003 Physiol. Rev. 83 (3), 835-870; Barrientos et al., 2008 Wound Repair Regen. 16 (5), 585-601; D'Alessio et al., 2008 J. Cell Sci. 121 (Pt 23), 3922-3932; McDowall et al., 2008 Cytokine Growth Factor Rev. 19 (5-6), 415-426; de Kretser et al., 2012 Mol. Cell. Endocrinol. 359 (1-2), 101-106; Pakyari et al., 2013 Adv. Wound Care (New. Rochelle. 2 (5), 215-224; Rao et al., 2013 Oncogenesis 2, e5; Liu et al., 2016 Sci. Rep. 6, 32231; Grada and Falanga, 2018 “Stem cell therapies for wound healing,” in Chronic wounds, wound dressings and wound Healing, Recent clinical techniques, results, and research in wounds, 301; Cooper et al., 2018 J. Invest. Dermatol. 138 (11), 2470-2479; Ferreira and Gomes, 2018 Bioeng. (Basel) 6 (1), 4; Mahmood et al., 2018 Front. Oncol. 8, 24; Park et al., 2018 Mol. Ther. 26 (2), 606-617; Ahangar et al., 2020 Int. J. Mol. Sci. 21 (19), 7038; Huang et al., 2020 Tissue Eng. Part B Rev. 26 (6), 555-570; Johnson et al., 2020 Biomedicines 8 (5), 101; Ham et al., 2021 Exp. Dermatol. 30 (3), 402-408).

Interestingly, Insulin and VEGF concentrations were higher in hypoxic CM than in normoxic CM. It is known that Insulin and VEGF expressions are up-regulated in hypoxia and stimulate angiogenesis by promoting endothelial cell survival, proliferation, migration, and remodeling (Werner and Grose, 2003, supra; Das et al., 2010 Tissue Eng. Part B Rev. 16 (2), 159-168; Escudero et al., 2017 Front. Physiol. 8, 204; Ferreira and Gomes, 2018, supra; Grada and Falanga, 2018, supra; Ahangar et al., 2020, supra; Huang et al., 2020, supra). Previous studies also demonstrated that hypoxic CM enhanced wound closure compared with normoxic CM and found that hypoxic CM contains higher amounts of proteins, e.g., bFGF, VEGF, IL-6, and TGFβ-1 (Chen et al., 2014). Based on these results with a given concentration of each protein, we developed a specific combination of recombinant proteins (CMF; ANG-1, FGF-7, Follistatin, HGF, IL-6, Insulin, TGFβ-1, uPAR, and VEGF) for wound healing therapy.

Several studies showed significantly different secretome profiles depending on cell sources (Kehl et al., 2019 npj Regen. Med. 4, 8.; Matz et al., 2020 J. Tissue Eng. Regen. Med. 14 (10), 1394-1402; Shin et al., 2021 Int. J. Mol. Sci. 22 (2), 845). For instance, our previous study investigated the secreted proteins in the CM of human cells derived from four different sources, hPSC, AFSC, adipose-derived stem cells (ADSC), and HUVEC, showing different protein secretion depending on cell sources (Matz et al., 2020, supra). Interestingly, a large number of angiogenic and antiapoptotic proteins with high concentrations were secreted from hPSC compared to the others (e.g., IL-6, HGF, and FGF-7). Our previous study showed that hPSC secreted follistatin, uPAR, and HGF with high concentration (Kim et al., 2022 Front. Bioeng. Biotechnol. 10, 848679). Another study by others also showed that the major proteins in hPSC-CM included HGF, TGFβ, and IL-6 (Shin et al., 2021, supra). The present study also obtained similar results as the proteins mentioned above were highly expressed in the hPSC-CM in this study.

The optimal CMF concentration was determined in terms of HDF growth. Our CMF concentration study showed that CMF increased HDF growth in a dose-dependent manner but not in high concentrations (>6×). We observed that the CMF treatment with high concentrations, especially CMF 10×, inhibited the HDF growth in hypoxia. It is plausible that this inhibitory effect is due to the multifunctional role of some proteins. For example, IL-6 and TGFβ-1 not only enhance cell survival and proliferation but also induce apoptosis depending on the environment (Moodley et al., 2003 Am. J. Respir. Cell Mol. Biol. 29 (4), 490-498; Sanchez-Capelo, 2005 Cytokine Growth Factor Rev. 16 (1), 15-34). As CMF at a concentration of 6× gave the best cell growth result, CMF 6× was used for further studies. We then compared the HDF growth between nine proteins combination (CMF) and individual proteins. The data showed that the CMF was more effective on HDF growth than the individual proteins.

This study demonstrates that the CMF is as effective as the stem cell-based therapies for accelerating wound healing. CMF at the optimized concentration (6×) supported HDF growth and had similar effects as CM (6×) and hPSC co-culture. In addition, the CMF showed similar anti-apoptosis and wound closure effects to the CM. Interestingly, the CM and CMF did not promote HDF proliferation but did reduce apoptosis.

We also demonstrated the angiogenic effects of the CMF. As discussed above, wound sites are often hypoxic due to the loss of blood supply caused by trauma, burn, or injury. Furthermore, the proliferation phase of wound healing is highly metabolic with increased demand for oxygen and nutrients, requiring re-epithelialization, granulation tissue formation, and scarless healing (Landen et al., 2016 Cell. Mol. Life Sci. 73 (20), 3861-3885). Therefore, restoring blood supply to the wound sites through promoting angiogenesis is critically important in improving wound outcomes. The in ovo CAM assay results showed that the CMF enhanced new vessel formation from pre-existing vessels. We also investigated the effect of CMF on HUVECs. The CMF treatment increased HUVEC growth compared with SFM; however, the CMF was not as effective as the CM. It is speculated that several proteins present in the CM but not included in the CMF composition also affected HUVEC growth, e.g., angiogenin, SDF-1, IL-8 (Gillitzer and Goebeler, 2001 J. Leukoc. Biol. 69 (4), 513-521; Tello-Montoliu et al., 2006 J. Thromb. Haemost. 4 (9), 1864-1874; Deshane et al., 2007 J. Exp. Med. 204 (3), 605-618). Other proteins not included in this study will need to be tested in subsequent studies to improve the angiogenic effects. In addition, the angiogenic effects of the CMF need to be further evaluated using various angiogenesis assays.

Our strategy of using recombinant proteins (CMF) having similar therapeutic effects to cell-based therapy provides many advantages over cell-based therapies. This treatment modality uses commercially available recombinant proteins with defined composition and concentrations. Thus, consistent product quality can be achieved, and safety and regulatory concerns can be minimized and further verified. This strategy can save time, effort, and resources by removing massive cell culture and CM collection processes. More importantly, our treatment modality as an off-the-shelf product facilitates immediate care when desired for traumatic wounds and burns. In addition, the major role of these proteins is promoting growth/proliferation, preventing apoptosis of several cell types not limited to skin cells, and promoting angiogenesis, which is critical for several types of tissue regeneration. Therefore, we expect this treatment modality to be applied to several tissue types for regeneration and reconstruction. For instance, we previously developed a specific combination of recombinant proteins by hPSC-CM analysis to treat kidney diseases (Kim et al., 2022 Front. Bioeng. Biotechnol. 10, 848679). We identified five major proteins (Follistatin, uPAR, ANGPTL4, HGF, and VEGF) that promote kidney repair in the hPSC-CM. The intrarenal delivery of these proteins ameliorated renal damage and accelerated renal function recovery in a rat acute kidney injury model. The treatment effects were similar to the CM treatment. These results indicate the potential of using recombinant proteins identified by CM analysis as an alternative to the CM to treat damaged or injured tissues.

Additional Studies The treatment effects of CMF are validated and optimized with other endothelial cells and also other cell types, e.g., keratinocytes and immune cells. For efficient delivery of recombinant proteins in vivo, a controlled delivery system is used, and in vivo feasibility and efficacy of the CMF combined with the delivery system is investigated in an animal model of full-thickness skin wounds.

### Example 2: Delivery of CMF with Alginate-Gelatin Hydrogel Carrier

To deliver CMFs, we prepared an alginate-gelatin hydrogel combination and investigated their physical characteristics (water absorption, stability). The biological activity and the release profile of the hydrogel delivery system were determined in vitro. To determine the therapeutic efficacy of tailored CMFs, we implanted and evaluated CMFs-loaded hydrogel in a rodent full-thickness wound defect model.

We observed that the CMF-loaded alginate-gelatin hydrogel provides appropriate physical characteristics, is non-cytotoxic, and provides controlled release of the loaded model drug (Dextran). The therapeutic efficacy study in vivo showed that treatment with CMF significantly accelerated wound closure, tissue formation, and matured re-epithelialization. In addition, CMF-loaded hydrogel demonstrated an increased matured vessel diameter and basket-weave collagen network, which is phenotypically similar to native skin tissue.

These results suggest that the CMF-loaded alginate-gelatin combination gel carriers are a promising and effective therapeutic solution for full-thickness wound healing applications.

### Materials and Methods

### Preparation of Alginate-Based Hydrogel

Several sodium alginate-based hydrogels were prepared by mixing with various polymers. Sodium alginate (Sigma-Aldrich, St. Louis, MO) was dissolved in normal saline (0.9% NaCl) to make a 10 mg/ml solution. To generate various alginate-based composite hydrogels, the same amount of polymer (10 mg/mL), such as poly(vinyl alcohol) (PVA, Sigma-Aldrich, St. Louis, MO), Gelatin (Sigma-Aldrich, St. Louis, MO), poly(ethylene glycol) (PEG, Sigma-Aldrich, St. Louis, MO), Gellan Gum (Sigma-Aldrich, St. Louis, MO) was incorporated into alginate solution at a 1:1 ratio (final concentration, 2% alginate-polymer).

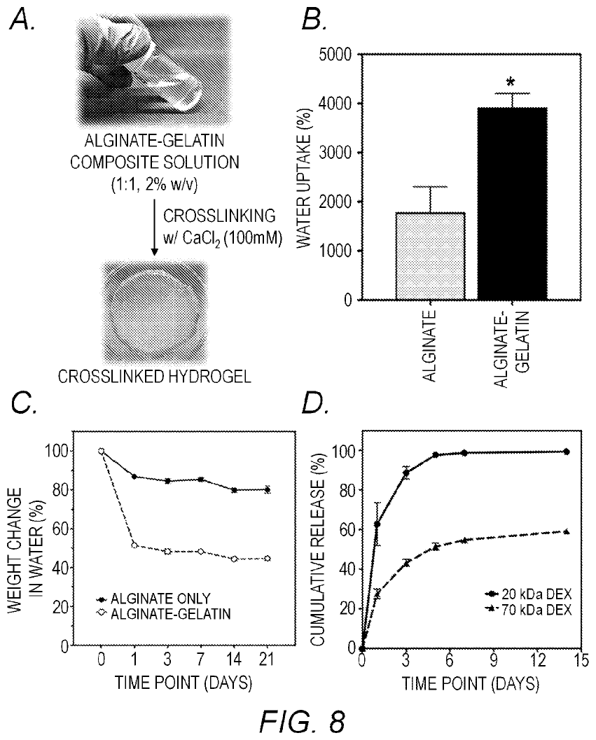

We prepared four different alginate-based hydrogel combinations: Alginate/PVA, Alginate/PEG, Alginate/Gelatin, and Alginate/Gellan Gum. All solutions were then sterile filtered using a 0.45 μm syringe filter. For crosslinking, 100 mM of Calcium Chloride solution (CaCl₂, Sigma-Aldrich, St. Louis, MO) was prepared by dissolving in normal saline then stored in a spray bottle. To form gels, the alginate and CaCl₂ solution were mixed by spraying CaCl₂ solution onto a thin layer of alginate solution applied on the wound bed, after which the gel began to crosslink within 1 minute (FIG. 8, Panel A).

### Water Uptake and Stability of Hydrogel

The water uptake (absorption) of the alginate-based hydrogel was examined by measuring the water absorption content. The hydrogels were weighed after thorough drying (Wdry) and then immersed in DI water. After 24 hrs, the hydrogels were removed from the water, wiped with tissue paper to remove excessive water, and immediately weighed again (Wwet). Water uptake was determined as follows:

$$Water uptake ( \% ) = ( Wwet - Wdry ) \times 100 / Wdry$$

To examine the stability of the alginate-based hydrogel, the hydrogels were immersed in water for 1, 3, 7, 14, and 21 days with continuous shaking. The hydrogel was removed from the water, dried to constant mass in a vacuum oven, and weighed (Wdry,final). The stability of the hydrogels was determined as follows:

$$Hydrogel stability ( \% ) = ( Wdry - Wdry , final ) \times 100 / Wdry$$

### In Vitro Biological Evaluations of Alginate-Gelatin Hydrogel

HPSCs isolated from donated human placenta were obtained from the Manufacturing Development Center at Wake Forest Institute for Regenerative Medicine. HPSCs (passage 6-7). The cells were cultured in HPSC growth media (HPSC-GM) containing 65% of Alpha Minimum Essential Medium (d-MEM; Gibco, Amarillo, TX), 17% Amniomax C100 Basal Media (Gibco), 15% Fetal Bovine Serum (FBS; Gibco), 2% Aminomax Supplement (Gibco), 1% Glutamax (Gibco), and 2.5 μg/ml Gentamicin (Gibco). Human dermal fibroblasts (HDFs) isolated from infant foreskin were cultured in Dulbecco's Modified Eagle Medium (DMEM; Cytiva; Logan, UT) containing 10% FBS and 1% Penicillin/Streptomycin (P/S; Fischer Scientific, Waltham, MA) (HDF-GM) (passage 5-7). The medium was changed every 2-3 days.

For cytotoxic evaluation, the alginate-based composite hydrogels (200 l) were placed at the center of sub-confluent HPSCs on 24-well plates. Cells cultured with growth media alone (no hydrogel) served as a negative control. A piece of Latex rubber served as a positive control (cytotoxic condition). The cell-hydrogel contact was maintained for 24 hours at 370 C in a 5% CO₂ atmosphere. After 24 hours, the cells cultured with the hydrogels were tested for cell viability using Live/Dead staining and an alamarBlue assay. Briefly, the cells were incubated in alamarBlue assay reagent (1:10 dilution) for 2 hours at 37° C. The incubated reagent was transferred to clear flat bottom 98 well plates, and fluorescence was measured at a wavelength of 560 and 590 nm (excitation and emission) (Spectra Max M5 multiplate reader, Molecular Devices, San Jose, CA) (n=3 per group and time point, duplicate measure).

For the cell proliferation assay within the gelatin-based hydrogel, HPSCs (5 million cells/mL) encapsulated into the hydrogel (2%) were incubated for 1, 3, and 7 days at 37° C., and the cell viability was assessed by CellTiter-Glo 3D (Promega) reagent following the manufacturer's protocol. At pre-determined time points, cell-encapsulated gels were transferred to the mixture of cell culture medium and CellTiter-Glo reagent solution (1:1), then incubated for 15 minutes at room temperature. Luminescence from each sample was measured through the plate reader (n=3 per group and time point, duplicate measure).

### Preparation of HPSC-Derived CM- and CMF-Loaded Hydrogel

HPSCs (10,000 cells/cm²) were plated in a 15 cm culture dish and cultured overnight in GM. The cells were re-fed with serum-free HPSC media (HPSC-SFM; HPSC-GM without FBS and Amniomax Supplement) and incubated in normoxia (21% O₂) and hypoxia (1% O₂) (Hypoxia C-chamber, Biospherix, Parish, NY) for 3 days. HPSC-CM was collected, centrifuged at 1500 rpm for 5 minutes, and the supernatant was collected to eliminate cellular debris. The HPSC-CM was concentrated using Amnicon Ultra-15 Centrifugal Filter Units with a molecular cutoff of 3 kDa. (Millipore Sigma, Germany). The concentrated HPSC-CM was frozen and stored at −80° C. until use. To prepare HPSC-CM- and CMF-loaded hydrogels, the 10× concentrated HPSC-CM (final 6×) and 9 protein cocktails (6×, CMF) were added to the alginate/gelatin composite solution (2 wt. %). Recombinant human Angiopoietin-1 (ANG-1; R&D Systems, Minneapolis, MN), Fibroblast growth factor-7 (FGF-7; R&D Systems), Follistatin (Sigma-Aldrich, St. Louis, MO), Hepatocyte growth factor (HGF; Sigma Aldrich), Interleukin-6 (IL-6; R&D Systems), Insulin (Sigma Aldrich), Transforming growth factor beta-1 (TGFβ-1; R&D Systems), Urokinase receptor (uPAR; R&D Systems), and Vascular endothelial growth factor (VEGF; R&D Systems) were used as a 9 recombinant protein cocktail. Based on the previous study, the composition and concentration of 9 recombinant proteins as a wound healing therapy was determined (Table 3).

TABLE 3
Stem cell-derived 9 recombinant proteins for wound healing

| | Concentration |
|---|---|
| Recombinant Proteins | (ng/mL) |
| Angiopoietin-1(ANG) | 6.3 |
| Fibroblast growth factor-7 (FGF-7) | 2.1 |
| Follistatin | 16.5 |
| Hepatocyte growth factor (HGF) | 25.0 |
| Interleukin-6 (IL-6) | 8.0 |
| Insulin | 25.0 |
| Transforming growth factor beta-1 (TGFβ-1) | 62.2 |
| Urokinase receptor (uPAF) | 7.8 |
| Vascular endothelial growth factor (VEGF) | 3.5 |

### Release Kinetic Study

Growth factor release kinetics were evaluated using FITC-dextran loaded hydrogel as a substitute for CMFs. The hydrogels were loaded with different molecular weights of FITC-dextran (20 kDa, 70 kDa) to allow reliable approximation of the broad range of molecular weight for CMFs (e.g., VEGF-20 kDa, FGF −70 kDa). FITC-dextran-loaded alginate/gelatin combination hydrogels were placed in transwell inserts in 24-well plates filled with PBS (release buffer) and stored at 37° C. At days 1, 3, 5, 7, and 14, the amount of the FITC-dextran released into the supernatant was determined by measuring the fluorescence intensity at a wavelength of 490 and 520 nm (excitation and emission) (Spectra Max M5 multiplate reader, Molecular Devices, San Jose, CA) (n=3 per group and time point, duplicate measure). A calibration curve prepared by measuring the intensities of known concentrations was used to calculate the concentration of the released FITC-dextran from the hydrogel.

### Full Thickness Wound Treatments

The therapeutic efficacy of the nine factors-loaded alginate/gelatin composite hydrogel onto wounds was evaluated by creating a circular shape (diameter—12 mm) full-thickness excisional wound on the back of athymic nude (Nu/nu) mice (Charles River Laboratories, Wilmington, MA). All animal procedures were performed according to protocols approved by the Wake Forest University Health Sciences Animal Care and Use Committee. The full-thickness defect was made by surgical removal using a 12 mm biopsy punch. Two wounds per animal were made along the dorsal lateral surface centered, 1-2 cm from the midline, and positioned near the intersection of the thoracic and lumbar regions (<6% of the total skin surface area). Wounds were filled with either some variant of the alginate/gelatin hydrogel or left to heal on their own. The hydrogels were cross-linked with a calcium-chloride solution (100 mM) which was applied by way of a spray bottle. Wounds served as the unit of measure for this study and each wound was randomly assigned to one of the treatments, which included: (1) defect only, (2) hydrogel only, (3) hydrogel plus CM, (4) hydrogel plus HPSCs, and (5) hydrogel plus CMF. Some animals studied at 1, 2, 3, 4, 6, or 8 weeks postoperatively. The first group did not receive treatment; instead, only a standard wound dressing was applied to cover the wounds (Defect only). The second group received alginate/gelatin (2%) hydrogel only (Gel only). The third group was HPSC-CM-loaded (6×concentration) hydrogel (CM). The fourth group received HPSCs-encapsulated (15 million cells/mL) hydrogel (PSCs). The fifth group was treated with 9 recombinant proteins cocktail-loaded (6× concentration) hydrogel group (CMFs). All groups were bandaged by utilizing a closely adhering covering with loban (3M, St. Paul, MN), which allows for the wound bed to maintain its moisture content. A thin strip of tape was laid across the animal's abdomen to secure the bandage, and vetwrap was wrapped around the animal to provide padding. The ends of the bandage were cut and splayed apart to be fastened onto the fabric, which prevents slippage of the bandage along the animal's longitudinal axis. Finally, a thin layer of tape was used to provide a water-resistant cover for the entire bandaging system. The mice were monitored daily for post-operative recovery, and the wounds were inspected daily with the outer bandages changed at least once a week or more frequently, if needed. Mice were euthanized at their experimental endpoint, and the wounds with their surrounding tissues harvested.

### Gross Evaluation and Histological Analyses

Photographs of the wound were taken for each group at the time of harvest. The photographs were taken using a 12-megapixel lens and sterile ruler/mask. The area of open wounds, which were shiny and dark red in color, was measured from the photographs using ImageJ software (National Institutes of Health). Skin samples retrieved from the mice were fixed in 10% phosphate-buffered formalin overnight at room temperature. Samples were dehydrated in a graded series of ethanol concentrations, embedded in paraffin, and cut into 5 m sections. Slides were then stained with hematoxylin and eosin, Masson's trichrome, and picrosirius red for histology, and imaged by light microscopy. The picrosirius red-stained sections required an additional polarized filter for optimal imaging. The stained Images were then examined to determine the degree of wound healing, re-epithelialization, and collagen structure/alignment, and compared with normal undamaged mouse skin sections.

### Picrosirius Red Collagen Fiber Structure Analysis

Once picrosirius red images were captured, they were analyzed with the CTFIRE and CurveAlign programs, designed by the Laboratory for Optical and Computational Instrumentation, University of Wisconsin-Madison. Default settings were used, and data were imported into MATLAB for analysis and graphing. CTFire was used to overlay each collagen fiber which were measured for width and length (~300 fibers per image). The resultant mean collagen fiber length in pixels, standard deviation, and the sample size were then statistically analyzed using GraphPad Prism software. Similarly, CurveAlign was used to overlay each collagen fiber (~300 fibers per image), which was then converted into a direction heat map, allowing for quantification of the coefficient of alignment; with ‘zero’ being no alignment (like uninjured skin collagen), and ‘1’ being complete alignment (more scar-like). The coefficient of alignment for each group was then analyzed using GraphPad Prism software.

### Immunohistochemistry

Immunohistochemical staining (IHC) staining with alpha-smooth muscle actin (α-SMA) and Ki-67 was used to visualize matured blood vessels and cellular proliferation, respectively. For IHC, all incubations were carried out at room temperature unless otherwise stated. Slides were warmed at 60° C. for 1 hour to increase bonding of the tissue sections to the slides. Antigen retrieval was performed on all slides and achieved with incubation in Proteinase K (Dako, Carpinteria, CA) for 5 minutes. Sections were permeabilized by incubation in 0.1% Triton-X for 5 min. Non-specific antibody binding was blocked by incubation in Protein Block Solution (Abcam) for 15 minutes. Sections were incubated for 60 minutes in a humidified chamber with the primary antibodies α-SMA (1:100, Cat. #ab5694, Abcam), or Ki67 (1:100, Cat. #ab833, Abcam) in antibody diluent (Abcam). Following primary incubation, slides were washed 3 times in PBS for 5 minutes. Sections were then incubated for 20 minutes with TexasRed anti-rabbit (T11000; Vector Laboratories, Burlingame, CA) secondary antibody solution at a 1:200 dilution. After washing, sections were washed in PBS, counterstained with 4′,6-diamidino-2-phenylindole (DAPI, Life Technologies, Eugene, OR), and coverslipped with Prolong Gold Antifade (Invitrogen, Carlsbad, CA). Positive and negative controls were performed in the same manner as described above. All samples were imaged using a Leica DM 4000B upright microscope with fluorescence at 594 (TexasRed) and 359 nm (DAPI).

### Statistical Analysis

Statistical analysis of wound healing data was performed using GraphPad Prism (Graphpad Software Inc.) and involved a two-way ANOVA with comparisons between each treatment with each time-point. For multiple comparisons, a Tukey test was used and a significance and confidence interval of 0.05 and 95%, respectively. All data are presented as mean±standard deviation (n=5).

### Results

### Physical Characterization of Alginate-Based Composite Hydrogel

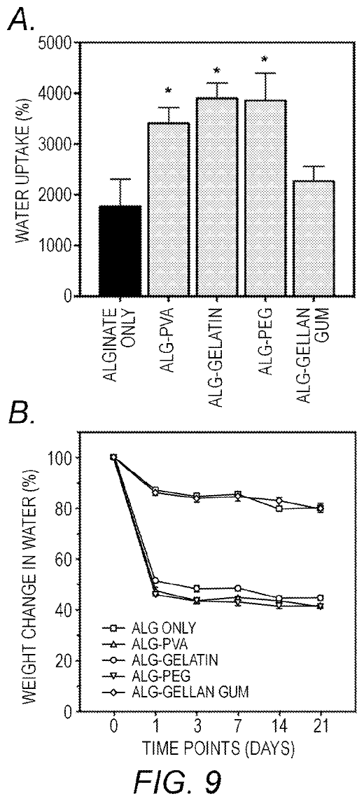

FIG. 8, Panel A depicts the cross-linking process of alginate/gelatin composite hydrogel by adding CaCl₂ and shows a visual representation of the cross-linked hydrogel. Since both alginate and gelatin are water-soluble polymeric materials, these polymers were well-mixed at a 1:1 ratio in an aqueous solution. The solution was transparent without any insoluble particles. The surface started to solidify immediately, where a transparent hydrogel without any morphological changes was obtained. We confirmed that the center region of hydrogel was fully solidified after 10 min crosslinking procedure. The amount of water uptake of alginate only and alginate/gelatin hydrogel is shown in FIG. 8, Panel B. Although the same concentration (2% w/v) was applied for both hydrogels, the water uptake of the alginate/gelatin hydrogel group (3903.3±301.1%) was significantly higher (*p<0.05) than that of the alginate-only hydrogel group (1767.8±539.8%). The weight of the alginate/gelatin hydrogel group dramatically decreased (51.4±0.3%) even after day 1 submersion in DI water, compared to the alginate-only hydrogel group (86.8±0.5%). However, the alginate/gelatin hydrogel group showed slight weight loss but remained stable for 21 days (44.6±0.8%) (FIG. 8, Panel C). We also investigated other alginate-based hydrophilic combinations. Those combinations showed higher water uptake properties and similar long-term stabilities in the DI water (FIG. 9).

The release of FITC-labeled dextrans at two different molecular weights (20 kDa and 70 kDa) from the alginate-gelatin hydrogels was used to characterize the release kinetics. FIG. 8, Panel D shows the cumulative release profile of FITC-dextran for 14 days. The FITC-dextran with a 20 kDa molecular weight showed a burst release after 1 day (62.8±10%), and almost all of the loaded dextran had been released from the hydrogel by day 7 (98.7±0.22%). By contrast, the FITC-dextran with a 70 kDa showed a slow-release pattern of 27.5±2.46% after 1 day followed by a continuous release up to 59±1.28% after 14 days (FIG. 8, Panel D). The larger molecular weight dextran released more slowly than the small molecular weight of dextran. This is most likely due to the entrapment of the larger size of drug into the hydrogel network.

### In Vitro Biological Activity Test of Alginate-Based Composite Hydrogel

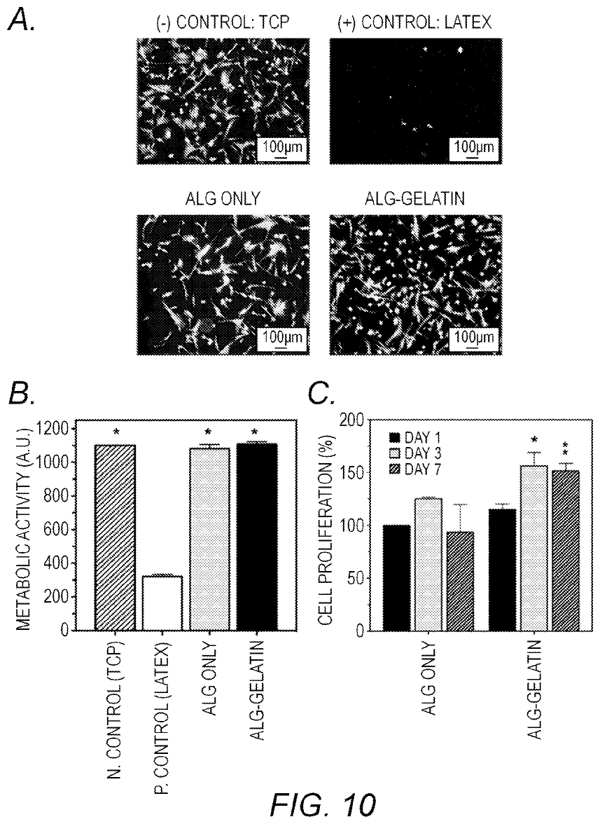

The biological activity test indicated that both the alginate-only hydrogel and the alginate/gelatin hydrogel were not cytotoxic to HPSCs, as the metabolic activity of cells cultured with the hydrogels appeared nearly identical to that seen in samples of cells grown in 2D tissue culture plate (FIG. 10, Panel B). Furthermore, visual observation showed that all experimental groups were similar in live/dead assay (green-live cells, red-dead cells) and cell morphology, while the latex rubber as positive control showed significantly lower cell viability at 24 hours (FIG. 10, Panel A).

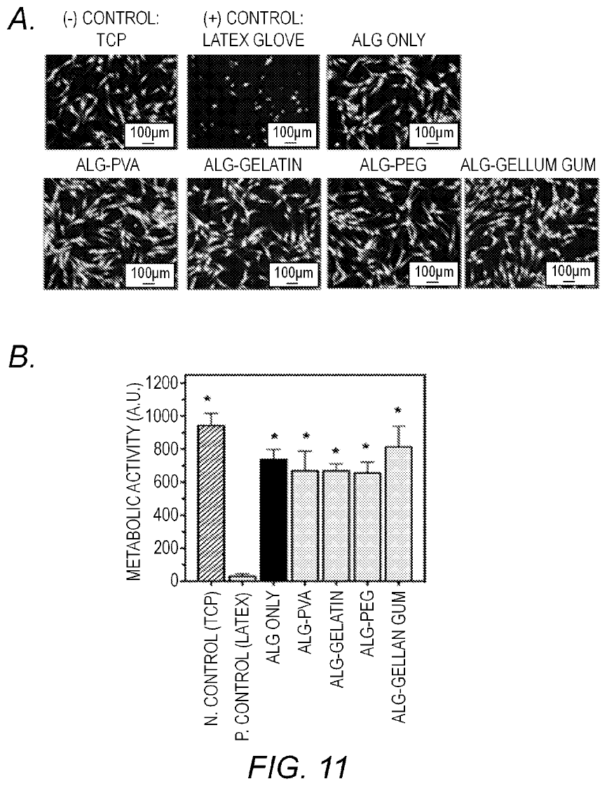

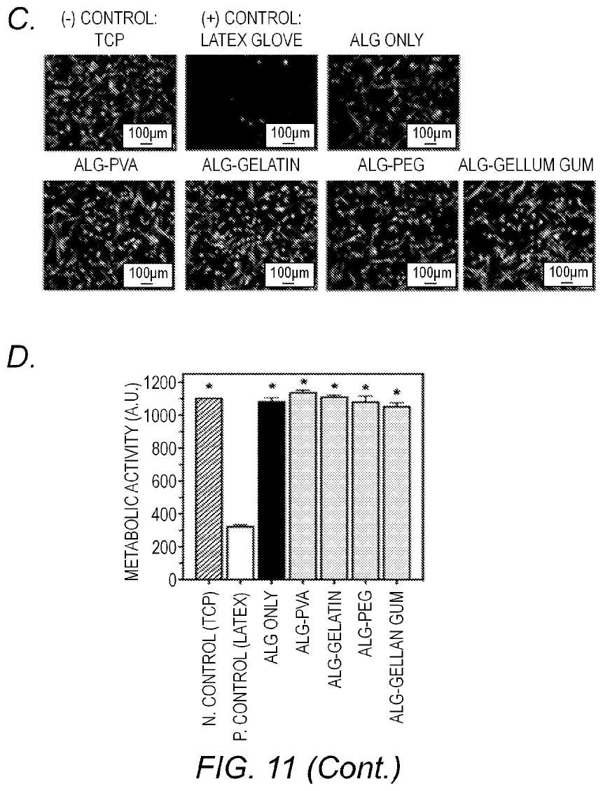

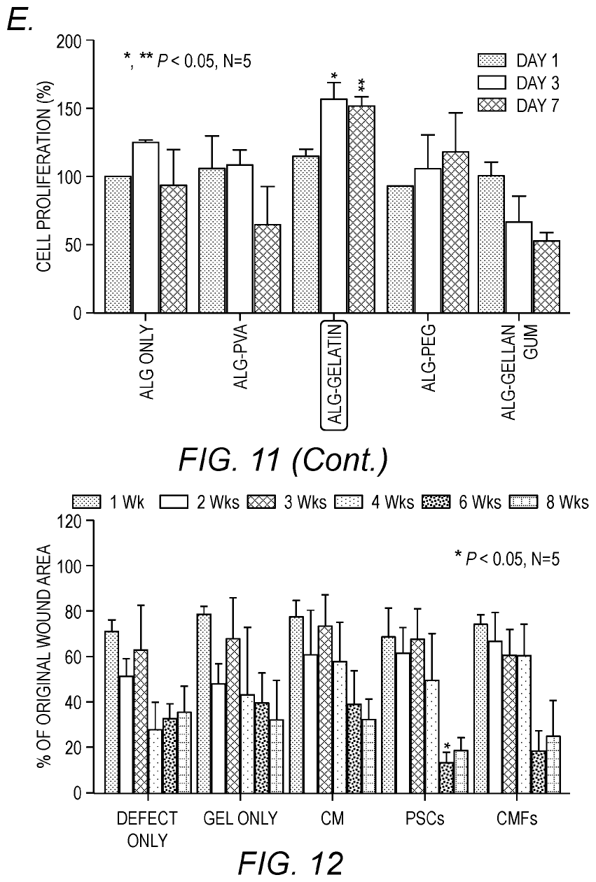

HPSCs proliferation within the hydrogels demonstrated that cell viability was significantly increased in the alginate/gelatin hydrogel at 3 days and 7 days post-encapsulation, compared to the alginate-only group. However, there was no statistical increase at 7 days when compared to 3 days (FIG. 10, Panel C). The results showed that the gelatin could enhance cell proliferation while alginate had poor cell proliferation. Other hydrogel combinations showed decreased cell proliferation and no statistical difference in metabolic activity over 7 days (FIG. 11). Thus, the alginate-gelatin hydrogel was selected for further in vivo study.

### Wound Closure and Re-Epithelialization

Digital images of full-thickness wound closure were taken. At each time point, no wound infection or skin irritation was noted on any treatment group. The alginate/gelatin hydrogel provided full coverage over the wound area and formed a tight seal at the edges of the wound defect. Gross observation revealed that every alginate/gelatin hydrogel treatment appeared to form a well-defined epithelial-like tissue organization (glossy transparent tissue layer) over the wound at 1 week. The PSC-encapsulated alginate/gelatin hydrogel treatment group and CMF-incorporated hydrogel treatment group demonstrated faster-wound closure than untreated defect only and all other treatment groups. The wound size in the PSC treatment group and CMF treatment group had a dramatically greater percentage of wound closure by the 6-week time point (13.1±5.0%, 18.5±8.8%, respectively), while the wound size in the untreated defect, gel only, and CM treatment group were 33.0±6.2%, 39.5±13.2%, 39.0±14.7%, respectively) (FIG. 12). At each time point, wound size in the PSC- and CMF-treated groups was not significantly different.

### Histological Analysis

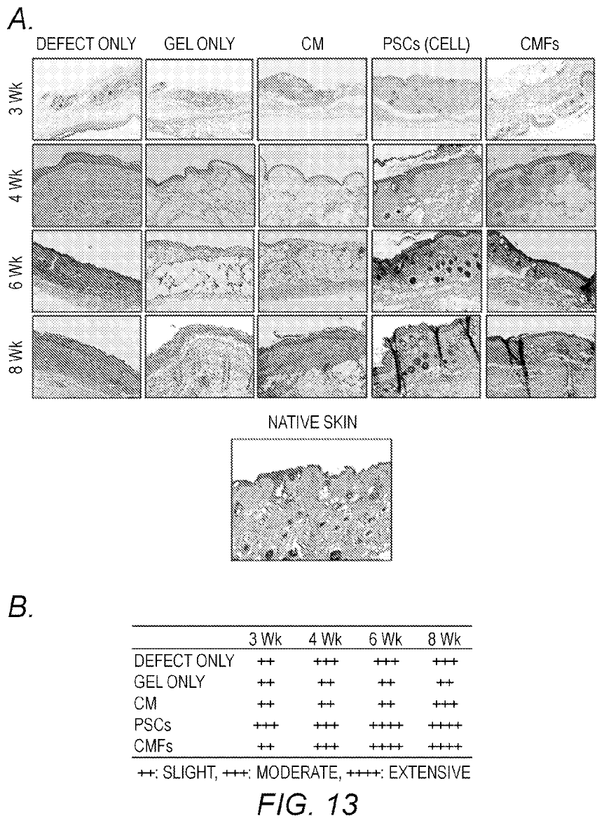

Representative images of sections stained with Hematoxylin and Eosin at all time points are shown in FIG. 13, Panel A. Healthy mouse native skin was used as a control for the analysis. At the 3-week time point, all groups showed increased cellularity and the presence of blood vessels in the wound defect area. Most wounds had an epithelial covering on the surface of the wound by the 4-week time point. The PSC- and CMF-treated group showed more cellular infiltration with granulation tissue formation, and more distinctive thicker epithelial tissue formation than the untreated control and other groups. Inconsistent epidermis coverage, less cellularity, and minimal dermal organization were observed in the gel only and CM treated group at all each time point. In all hydrogel implanted groups, we observed remaining gel particles in the wound defect area until the 6-week time point. However, there is no remaining gel particles in the PSC- and CMF-treated group at the 8-week time point, suggesting that increased cellularity accelerates the degradation of the gelatin portion of the hydrogels. By the 6-week and 8-week time point, the PSC- and CMF-treated groups appeared to have a more defined healthy dermis skin consisting of a large, organized fiber structure. Particularly, both the PSC- and CMF-treated groups had shown noticeably distinctive hair follicles regeneration, like native mouse skin. The summary table of wound healing assessment defined in terms of cellularity, tissue granulation, epidermal regeneration, angiogenesis, and hair follicle formation is shown in FIG. 13, Panel B.

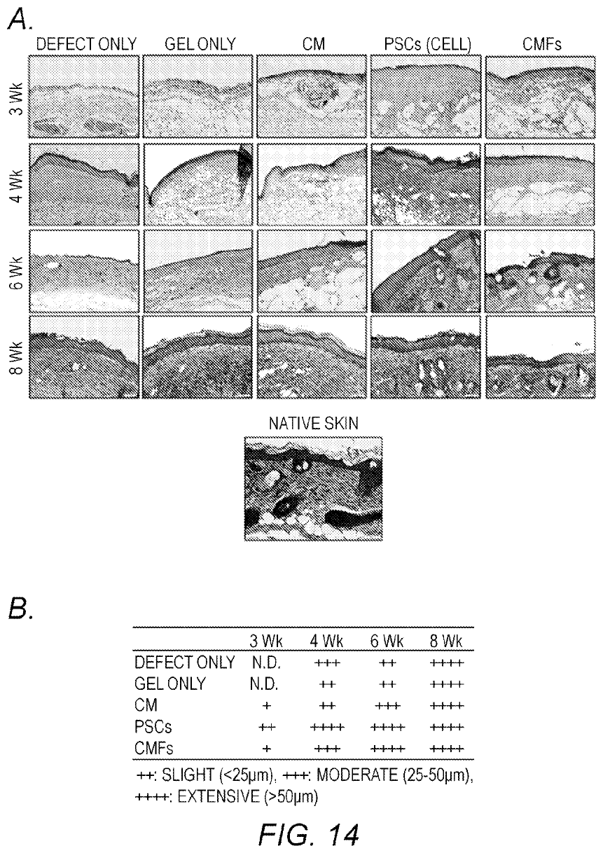

In FIG. 14, Panel A, Digital images of Masson's trichrome staining were shown to give an overview of keratin and collagen composition from the various wound treatments. Healthy mouse native skin was used as a control for the analysis. Masson's trichrome staining allows to identify multiple ECM components of the skin tissue. The representative image of stained healthy native skin is shown in FIG. 14. At 3-week and 4-week post-implantation, we observed highly disorganized collagen staining in the dermis with a disorganized epidermis layer and less cellularity in the untreated defect only group, gel only, CM-, and CMF-treated groups, but the PSC-treated group appeared intense keratin staining with moderate collagen staining, and high cellularity in the epidermis and dermis at the 4-week time point. At 6-week and 8-week post-implantation, the PSCs- and the CMFs-treated group showed a defined epidermis formation and highly organized dermis with increased cellularity, suggesting similar composition to healthy skin. The summary table of the re-epithelialization index defined in terms of keratin tissue formation is shown in FIG. 14, Panel B.

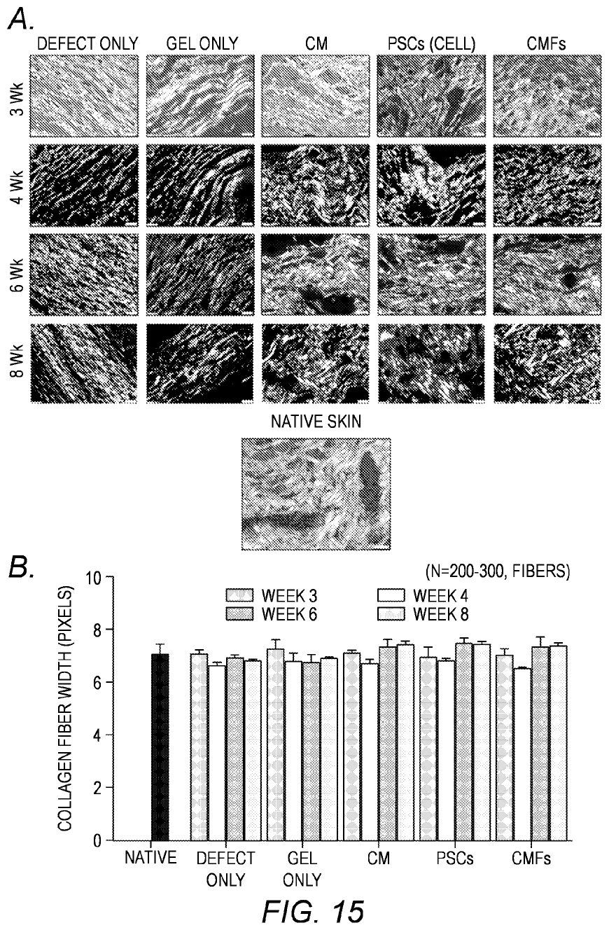

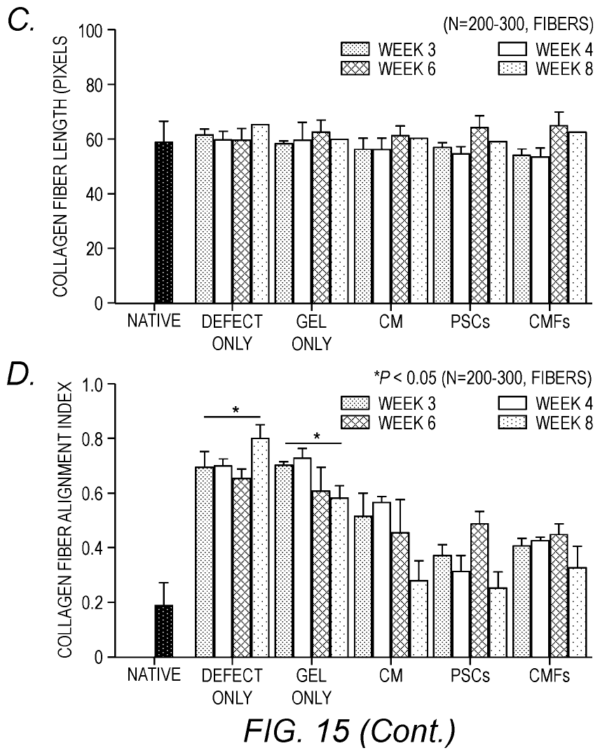

Picrosirius red staining was performed to analyze collagen fiber color, alignment, length, and width under polarized light. Healthy mouse native skin was used as a control for the analysis. Healthy skin showed strong staining of orange color, indicative of an organized network of mature collagen fibers in a basket weave orientation (FIG. 15), while parallel orientation indicates scarred or fibrotic tissue. We found that CM-, PSC-, and CMF-treated wounds had strong, orange-colored collagen with a basket weave orientation, indicating normal healing at the 6-week and 8-week time points (FIG. 15, Panel A and D, alignment index, less than 0.5). In contrast, the untreated control and gel-only group were mostly composed of collagen fibers aligned in parallel, indicating fibrosis or scarred tissue (FIG. 15, Panel D, alignment index, more than 0.8). There was no statistical difference in collagen fiber length and width between groups at all each time point (FIG. 15, Panel B and C).

### Immunohistochemistry

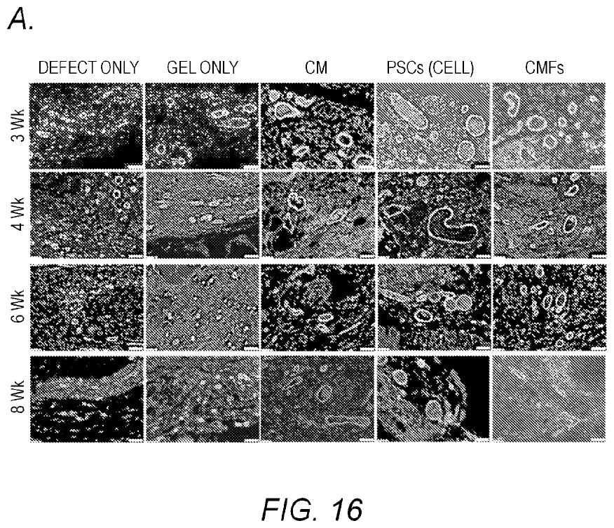

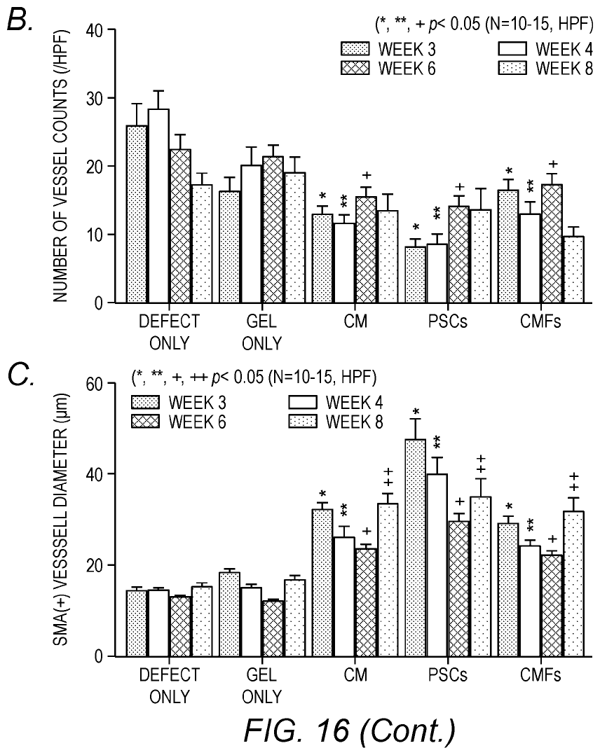

To evaluate the formation and maturation of wound vasculature, α-SMA immunostaining was performed (FIG. 16). We identified a large size of α-SMA-positive matured blood vessels form in the CM-, PSC- and CMF-treated groups at all time points (FIG. 16, Panel C). In contrast, the untreated defect only and hydrogel only treated group showed large numbers of small blood vessels, suggesting that these tissues were undergoing delayed wound healing (FIG. 16, Panel B).

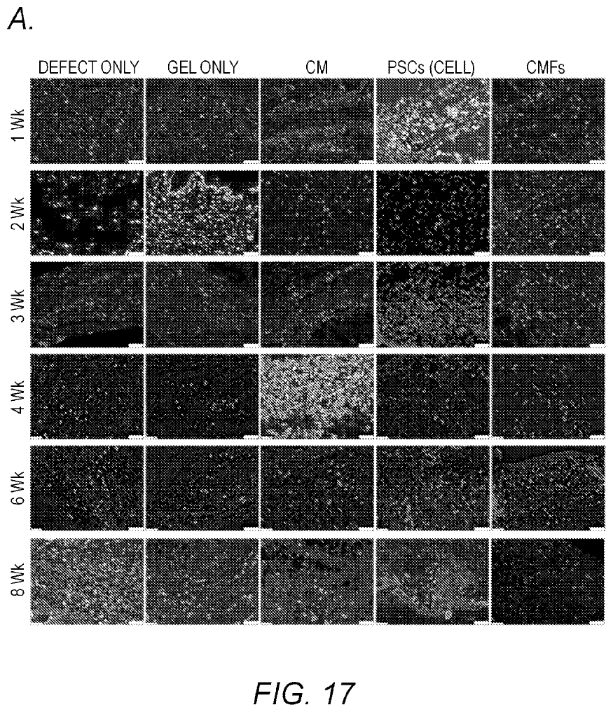

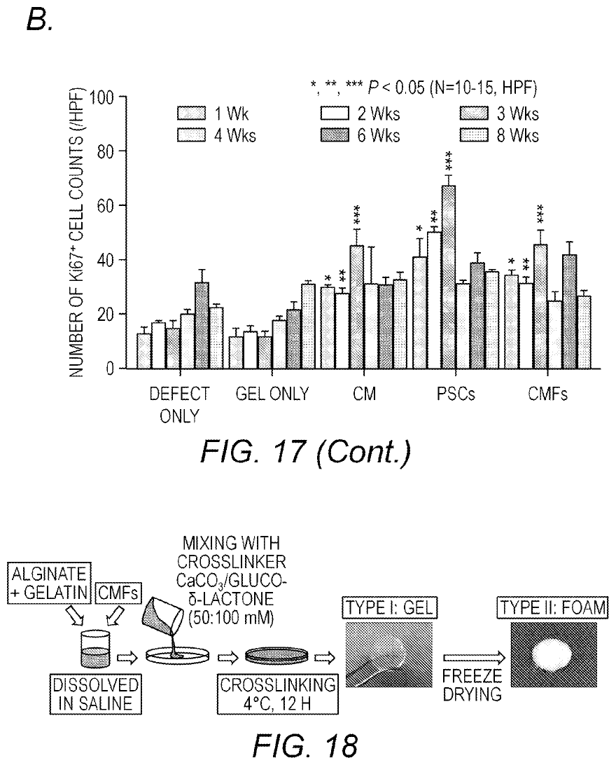

The presence of proliferating cells within the wound was analyzed by immunostaining with Ki67. The proliferating cells were steadily increased up to 3-weeks in the CM-, PSC-, and CMF-treated wound groups compared to untreated and hydrogel only group; however, the number of proliferating cells was not significantly increased in all groups, suggesting that rather than proliferating, these cells were contributing skin tissue formation and maturation (FIG. 17).

### DISCUSSION

This study aimed to develop a therapeutic option using CMFs based on HPSC-derived CM for wound healing therapy. To deliver CMFs, PSCs, and HPSC-CM, we utilized the alginate-gelatin hydrogel combination. Alginate is a natural polysaccharide obtained from brown seaweed. It has been extensively investigated for various biomedical applications, including wound healing and drug delivery due to its favorable properties, such as non-toxicity, biocompatibility, non-immunogenicity, affordability, and high absorption capacity. In addition, alginate is characterized by moisture control and anti-bacterial activity. Thus, it has been particularly attractive in wound healing applications.

Currently, there are many different forms of commercially available alginate-based wound dressings in clinical application, such as Algisite* (Smith & Nephew, London, UK), Algicell© (Integra LifeScience, Princeton, NJ), and Nu-Gel© (3M, St. Paul, MN). The alginate can be crosslinked by various crosslinking methods. Conventionally, the crosslinked-alginate hydrogel can be simply prepared by the addition of divalent cations (i.e., Ca²⁺). Calcium chloride (CaCl₂) is the most frequently used crosslinking agent for alginate hydrogel. Due to their structural stability and good water absorption properties by crosslinking, the alginate hydrogel allows extensive applications in delivering cells and bioactive agents, including small molecules and proteins.

From these unique properties of alginate, we developed the alginate-based hydrogels to deliver cells and CMFs. We first investigated their physical characteristics and biological activities in vitro. To consider an easy application to the wound bed, the alginate/gelatin hydrogel was crosslinked by spraying CaCl₂ solution onto the applied thin layer of the hydrogel solution surface. As expected, the hydrogel precursor could be fully gelatinized within several seconds by diffusion of calcium ions. Water uptake results showed that the amount of water in the alginate/gelatin group was significantly increased, compared to the alginate-only (2%) hydrogel group. It is indicated that the hydrophilic characteristic of gelatin provided more water absorption properties to the hydrogel. Although the alginate/gelatin hydrogel was initially less stable after submersion in DI water than the alginate-only hydrogel, it was fairly stable up to 21 days. While not wishing to be bound by theory, we hypothesize that the higher molecular weight of long chains of the gelatin might be entrapped in the alginate network structure for a long period.

In vitro biological activity study results showed that the alginate/gelatin combination enhanced cell proliferation when the HPSCs are encapsulated into the hydrogel, compared to the alginate-only hydrogel. Gelatin polymer derived from collagen contains important binding moieties for cell attachment that affects cell adhesion, proliferation, migration, and differentiation. Other hydrogel combinations showed decreased cell proliferation and no statistical difference for 7 days (FIG. 11, Panel C). Thus, the alginate-gelatin hydrogel was selected for further in vivo study as a delivery system for cells (positive control) and CMFs.

The release of FITC-labeled dextrans with two different molecular weights (20 kDa and 70 kDa) from the alginate-gelatin hydrogels was modeled to characterize the release kinetics for the nine proteins CMFs. The FITC-dextran with a 20 kDa. molecular weight showed a burst release after only 1 day and had been completely released from the hydrogel by day 7. In contrast, the FITC-dextran with a 70 kDa size showed a controlled-release pattern until 14 days (FIG. 12), suggesting the entrapment of the higher molecular weight dextran in the hydrogel network.

To evaluate the therapeutic feasibility of using the CMFs to accelerate wound healing, we compared the alginate/gelatin hydrogel with CM, PSCs, and CMFs in a rodent full-thickness wound defect model. Histological assay results showed that both PSC- and CMF treated groups were more effective on cellular infiltration with granulation tissue formation and more distinctive thicker epithelial tissue formation than the untreated control and other groups. Particularly, both groups had shown noticeably distinctive hair follicle regeneration, like native mouse skin at a later time point. In addition, we found that CM-, PSC-, and CMF-treated wounds showed more matured large diameter vessels and had thicker collagen fibers with a non-parallel basket weave orientation, which is phenotypically similar to native skin, while the parallel orientation of collagen fibers was observed in the control and gel only group, indicating scarred or fibrotic tissue formation around wound defect region.

In this study, we used HPSC, HPSC-CM, and 9 recombinant protein cocktails (ANG-1, FGF-7, Follistatin, HGF, IL-6, Insulin, TGFβ-1, uPAR, and VEGF) with specific compositions and concentrations which were identified for wound healing therapy. In our previous study, we developed a specific combination of recombinant 9 proteins forwound healing therapy. The data showed that these 9 proteins were highly expressed in HPSC-derived CM, and the use of the 9 protein combinations more enhanced cell growth and wound healing than the use of single proteins, indicating the orchestral effects.

A wide range of paracrine factors secreted by HPSC are known to play an essential role in the wound healing process. Werner et al., Regulation of wound healing by growth factors and cytokines. Physiol Rev. Jul 2003; 83(3):835-870.; Grada et al., Novel Stem Cell Therapies for Applications to Wound Healing and Tissue Repair. Surg Technol Int. Oct. 26, 2016; 29:29-37; Ahangar et al., Mesenchymal Stem Cell Secretome as an Emerging Cell-Free Alternative for Improving Wound Repair. Int J Mol Sci. Sep. 24, 2020; 21(19). FGF-7, VEGF, and TGFβ-1stimulates the migration of macrophages, dermal fibroblasts, and collagen deposition, and increase angiogenesis and granulation tissue for re-epithelialization. Jayaraman et al., Stem cells conditioned medium: a new approach to skin wound healing management. Cell Biol Int. Oct 2013; 37(10):1122-1128; Yamakawa et al., Advances in surgical applications of growth factors for wound healing. Burns Trauma. 2019; 7:10. HGF promotes keratinocytes survival, granulation tissue formation, and re-epithelialization. Li et al., HGF accelerates wound healing by promoting the dedifferentiation of epidermal cells through beta1-integrin/ILK pathway. Biomed Res Int. 2013; 2013:470418. Follistatin regulates hair follicle neogenesis47 and IL-6 enhances fibroblast proliferation during the wound healing process.

Although we demonstrated that the CMF therapy is effective as an alternative to stem cell-based therapies for accelerating wound healing, the CM hydrogel group did not show the acceleration of wound healing. These results suggest that some of the cytokines or the growth factors may have an inhibitory effect on the wound healing process. Sanchez-Capelo, Dual role for TGF-beta1 in apoptosis. Cytokine Growth Factor Rev. Feb 2005; 16(1):15-34.; Moodley et al., Inverse effects of interleukin-6 on apoptosis of fibroblasts from pulmonary fibrosis and normal lungs. Am J Respir Cell Mol Biol. Oct 2003; 29(4):490-49848,49.

This study used an alginate/gelatin combination for the delivery of stem cells and recombinant proteins. Gelatin hydrogel is generally enzymatically degraded in the body; however, alginate hydrogel cannot be fully eliminated from our body. It is inherently non-degradable in mammals due to a lack of the enzyme (i.e., alginase) needed. Rather, it can be dissolved by exchange reactions with monovalent cations (Na⁺) existing in the body fluids and release of the divalent cations (Ca²⁺) in the crosslinked gel. Lee et al., Alginate: properties and biomedical applications. Prog Polym Sci. Jan 2012; 37(1):106-126; Sahoo et al., Alginate and its application to tissue engineering. SNApplied Sciences. 2021/01/08 2021; 3(1):30. For this reason, we observed that small gel particles still existed underneath the wound bed, even at full wound closure. To overcome this limitation, chemically modified forms of alginate, which is degradable in physiological conditions, has been widely exploited in drug- or cell delivery applications. This biodegradable alginate may be used in physiological conditions for better wound healing. In addition, this study had only one concentration of CMFs (6×, based on the concentration in PSC-CM) which was optimized in our previous in vitro study. Even though the 6× concentration of CMFs showed a similar therapeutic effect to HPSC treatment, future studies may confirm the effectiveness with other concentrations to optimize the effectiveness in vivo.

### CONCLUSIONS

This study demonstrated the feasibility of a therapeutic option using CMFs based on HPSC-derived CM for wound healing therapy. The alginate/gelatin hydrogel formulation provided the optimal physical properties and cell compatibility for delivering PSCs and PSC-derived CMFs. The CMFs group, like the PSCs-encapsulated group, accelerated wound closure, tissue formation, and re-epithelialization in a rodent wound defect model. Wounds treated with the CM, PSCs, and CMFs showed more matured large diameter vessels and superior collagen remodeling (basket-weave collagen network), which is phenotypically similar to native skin tissue. These results suggested that the tailored CMF-loaded alginate/gelatin combination hydrogel can be a promising and effective therapeutic solution for full-thickness wound healing applications. Moreover, the use of CMF offers a product that seems to have similar bioactive properties of stem cells and their acellular byproducts but can be manufactured cheaper, easier, and with a higher degree of reliability than can be achieved by current cellular therapies. Therefore, this technology may provide an easier path through the translational pipeline with fewer FDA hurdles to cross.

### Example 3: Preparation of Alginate-Gelatin Hydrogel and Foam

To prepare alginate/gelatin-based composite hydrogels, sodium alginate (Sigma-Aldrich, St. Louis, MO) was dissolved in normal saline (0.9% NaCl) to make a 10 mg/ml solution. Then, gelatin (Sigma-Aldrich, St. Louis, MO) was incorporated into the alginate solution at a 1:1 ratio (final concentration, 2% alginate/gelatin). To prepare CMF-loaded hydrogels, 9 protein cocktails (6×, CMF) were added to the alginate/gelatin composite solution. The solution was then sterile-filtered using a 0.45 μm syringe filter. For crosslinking, 50 mM of Calcium carbonate (CaCO₃, Sigma-Aldrich, St. Louis, MO) and Glucono-S-lactone (GDL, Sigma-Aldrich, St. Louis, MO) were added to the alginate/gelatin solution. To form gels (Type 1), the solution was placed in the silicon mold and incubated at 4° C. for 12h. To obtain foam (Type 11), the crosslinked hydrogel was freeze-dried for 24 h (FIG. 18).

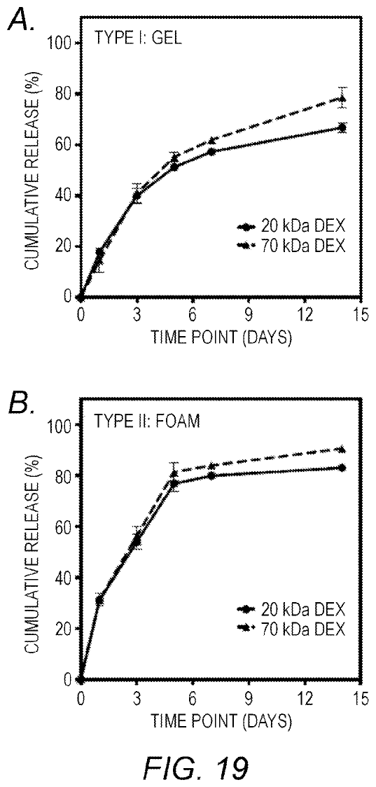

The release of FITC-labeled dextran at two different molecular weights (20 kDa and 70 kDa) from the alginate-gelatin hydrogels and foam was used to characterize the release kinetics. FIG. 19 shows the cumulative release profile of FITC-dextran for 14 days. The FITC-dextran with both 20 kDa. and 70 kDa. molecular weight showed a burst release after 5 days from both gel and foam types and the remained dextran had been slowly released. The FITC-dextran from the gel-type scaffold showed a slow-release pattern (60-70%), compared to the foam-type scaffold (over 80%) after 14 days.

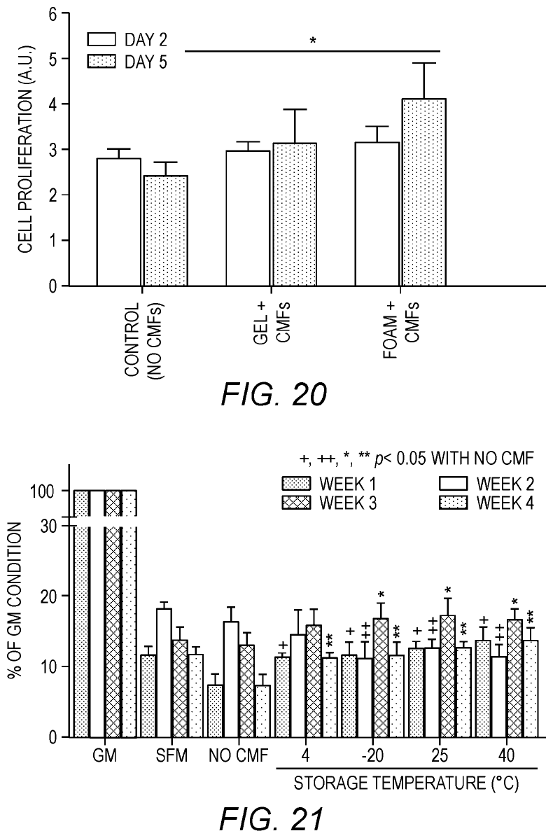

A biological activity test indicated that both the gel-type and the foam-type appeared nearly identical to that seen in samples of human dermal fibroblasts (HDFs) grown under control (no CMFs) group at day 2. However, HDFs proliferation under foam-type with CMFs demonstrated that cell proliferation was significantly increased at day 5, compared to the control group. These results are presented in FIG. 20.

To evaluate CMFs stability related to sample storage period and temperature, CMF-loaded foam-type scaffolds were stored between 1 week and 4 weeks. The storage temperatures were 4° C., 25° C., 40° C., and −20° C. The stability of CMFs levels of all samples was measured by the biological activity testing with culturing human dermal fibroblasts (HDF).

As shown in FIG. 21, the results demonstrated that all storage conditions with four different storage temperatures did not have a deleterious effect the biological activity for HDF proliferation. HDFs proliferation under foam-type with CMFs for long-term storage until four weeks demonstrated that cell proliferation was still significantly increased, compared to the control group (no CMF group).

At the 2-week time point, all groups showed increased cellularity and granulation tissue formation in the wound defect area based on sections stained with hematoxylin and eosin. Most wounds had an epithelial covering on the surface of the wound by the 4-week time point. By the 6-week and 8-week time points, the Gel+CMF and Foam+CMF groups appeared to have a more defined healthy dermis skin consisting of a large, organized connective structure. Particularly, both the Gel+CMF and Foam+CMF groups had shown noticeably distinctive hair follicle regeneration, like native skin. The summary table of wound healing assessment defined in terms of cellularity, tissue granulation, epidermal regeneration, angiogenesis, and hair follicle formation is provided in Table 4.

TABLE 4
Wound healing assessment: cellularity, tissue granulation, epidermal
regeneration, angiogenesis, hair follicle formation.

| | 1 W | 2 W | 4 W | 6 W | 8 W |
|---|---|---|---|---|---|
| Defect only | + | ++ | ++ | ++ | ++ |
| Gel only | + | ++ | ++ | ++ | +++ |
| Gel + CMFs | + | ++ | +++ | ++++* | ++++* |
| Foam only | ++ | ++ | ++ | +++ | +++ |
| Foam + CMFs | ++ | ++ | +++ | ++++* | ++++* |

+: Limited; ++: Slight; +++: Moderate; ++++: Extensive

### Example 4: Testing Alginate-Gelatin Foam with Stem Cell-Derived Recombinant Proteins for Wound Healing in a Preclinical Porcine Full-Thickness Skin Injury Model

The CMFs will be tested in a full-thickness skin wound model in a specific pathogen-free pig. All animal procedures will be performed according to protocols approved by the Wake Forest University Health Sciences Animal Care and Use Committee. Under anesthesia, the animals will be tattooed with six, 5×5 cm squares on the dorsum to denote the area of the excisional wound. The dorsal skin will be cleaned with water and soap and sterilized with β-iodine and 70% alcohol. For the creation of the defect, four areas of skin wound will be created by removing 5×5 cm of full-thickness skin in the dorsal midline along the thoracic and lumbararea. Incisions will be made along the wound edges with a surgical blade to the panniculus carnosus layer, and the overlying skin will be excised. There will be up to 4 wound beds per animal, each receiving a different treatment, which includes: (1) defect only, (2) graft only, (3) graft plus CMFs (low-dose), and (4) graft plus CMFs (high-dose). The first group will not receive treatment; instead, only a standard wound dressing will be applied to cover the wounds (defect only). The second group will receive only alginate-gelatin foam graft (graft only). The third group will be treated with low-dose of the CMF-loaded foam graft (CMF-L). The fourth group will be treated with high-dose of the CMF-loaded foam graft (CMF-H). After treatment application and for scheduled bandage changes, the wound area will be covered by dressing materials to provide a protective barrier.

The dressing materials will include topical antibiotic cream, Tegaderm, cast padding, cohesive bandaging, a stockinette, a protective plastic saddle, and a specially designed jacket to hold the bandage in place. Edges of the saddle will be covered with porous surgical tape along the front of the saddle to protect the pig's neck. Four Velcro® straps will be used to position a stretchable strap on the saddle to secure it to the pig. At each time point (1, 2, 4, 6, 8, and 12 weeks), the study will be terminated, and the wound areas will be harvested for analysis as indicated in Table 5.

TABLE 5
In vivo evaluation of wound healing and skin reconstruction of the alginate/gelatin
foam skin graft with CMFs in a full-thickness excisional wound model in the pig.

| Experimental group | Evaluation parameter | Evaluation method |
|---|---|---|
| No treatment (defect only) | Wound closure, contraction | Digital photographs |
| Alginate/gelatin skin graft only | Wound healing (cellularity, | Histology, e.g., H&E, MTS |
| Alginate/gelatin skin graft | tissue granulation, | |
| with low-dose CMFs (CMF-L) | epithelialization, hair follicle | |
| Alginate/gelatin skin graft | formation) | |
| with high-dose CMFs (CMF-H) | Collagen structure (fiber | Histology, e.g., Picrosirius Red |
| Timepoint: 1, 2, 4, 6, 8 and 12 | length, width, and alignment) | |
| weeks | Vascularization | Immunostaining, e.g., vWF, α- |
| Total 36 pigs (n = 6) | | SMA |
| | Cell proliferation | Immunostaining, e.g., Ki67, |
| | | PCNA |

### Gross Evaluation and Histological Examination

To evaluate the wound healing, photographs of the wound will be taken for each group at the time of harvest. The photographs will be taken using a 12-megapixel lens and sterile ruler/mask. Wound closure and epithelialization will be measured using ImageJ to determine the area of open wound and epithelialization, which can be separated by the color and texture of the healing wound area. Specifically, open wounds can be identified as being shiny and dark red, while the epithelium has a generally matte/opaque with a pink or white color tone. The wound contraction will be measured as the area inside the scared edge at each time-point, compared with the original open wound using ImageJ.

To perform the histological assay, skin samples retrieved from the animal at each time point will be fixed in 10% phosphate-buffered formalin overnight at room temperature. Samples will be dehydrated in a graded series of ethanol concentrations, embedded in paraffin, and cut into 5 μm sections. Slides will then be stained with hematoxylin, eosin, and Masson's trichrome for histology and imaged by light microscopy. The stained Images will be then examined to determine the degree of wound healing and re-epithelialization and compared with normal undamaged pig skin sections.

### Collagen Fiber Structure Analysis

To analyze collagen fiber structure, sectioned tissue samples on the slide will be stained with picrosirius red. The picrosirius red-stained sections will require an additional polarized filter for optimal imaging. Once picrosirius red images are captured, they will be analyzed with the CTFire and CurveAlign programs, designed by the Laboratory for Optical and Computational Instrumentation at the University of Wisconsin-Madison. Default settings will be used, and data will be imported into MATLAB for analysis and graphing. CTFire will be used to overlay each collagen fiber which will be measured for width and length. Similarly, CurveAlign will be used to overlay each collagen fiber, which will then be converted into a direction heat map, allowing for quantification of the coefficient of alignment, with ‘zero’ being no alignment (like uninjured skin collagen) and ‘1’ being complete alignment (more scar-like). The alignment coefficient for each group will then be analyzed using GraphPad Prism software.

### Immunohistochemistry

IHC staining with α-SMA and Ki-67 will be used to visualize matured blood vessels and cellular proliferation, respectively. For IHC, all incubations will be carried out at room temperature unless otherwise stated. Slides will be warmed at 60° C. for 1 hour to increase the bonding of the tissue sections to the slides. Antigen retrieval will be performed on all slides and achieved with incubation in Proteinase K (Dako, Carpinteria, CA) for 5 minutes. Sections will be permeabilized by incubation in 0.1% Triton-X for 5 min. Non-specific antibody binding will be blocked by incubation in Protein Block Solution (Abcam) for 15 minutes. Sections will be incubated for 60 minutes in a humidified chamber with the primary antibodies α-SMA (1:100, Cat. #ab5694, Abcam) or Ki67 (1:100, Cat. #ab833, Abcam) in antibody diluent (Abcam). Following primary incubation, slides will be washed 3 times in PBS for 5 minutes. Sections will be then incubated for 20 minutes with a secondary antibody solution. After washing in PBS, sections will be counterstained with DAPI (Life Technologies, Eugene, OR) and coverslipped with Prolong Gold Antifade (Invitrogen, Carlsbad, CA). Positive and negative controls will be performed in the same manner as described above. All samples will be imaged using a Leica DM 4000B upright microscope.

The foregoing is illustrative of the present invention, and is not to be construed as limiting thereof. The invention is defined by the following claims, with equivalents of the claims to be included therein.

1. A composition comprising:
i) from 0.5 or 1 ng/mL, to 4 or 5 ng/mL of recombinant fibroblast growth factor-7 (FGF-7);
ii) from 5, 6 or 8 ng/mL, to 28 or 30 ng/mL of recombinant follistatin;
iii) from 2, 3 or 4 ng/mL to 14 or 16 ng/mL of recombinant interleukin-6 (IL-6);
iv) from 15, 20 or 25 ng/mL to 110 or 125 ng/mL of recombinant transforming growth factor beta-1 (TGFβ-1); and
v) from 2 or 3 ng/mL, to 14 or 16 ng/mL of recombinant urokinase receptor (uPAR); and a carrier.

2. The composition of claim 1, wherein said composition further comprises:
vi) from 2 or 3 ng/mL, to 11 or 12 ng/mL of recombinant angiopoietin-1 (ANG-1);
vii) from 6, 7, 8 or 9 ng/mL, to 45 or 50 ng/mL of recombinant hepatocyte growth factor (HGF);
viii) from 6, 7, 8 or 9 ng/mL, to 45 or 50 ng/mL of recombinant insulin; and
ix) from 1 or 1.5 ng/mL, to 6, 8 or 10 ng/mL of recombinant vascular endothelial growth factor (VEGF).

3. The composition of claim 1, wherein said composition comprises:
i) from 1 to 3 ng/mL of recombinant fibroblast growth factor-7 (FGF-7);
ii) from 8 to 25 ng/mL of recombinant follistatin;
iii) from 4 to 12 ng/mL of recombinant interleukin-6 (IL-6);
iv) from 25 to 100 ng/mL of recombinant transforming growth factor beta-1 (TGFβ-1); and
v) from 3 to 12 ng/mL of recombinant urokinase receptor (uPAR).

4. The composition of claim 3, wherein said composition further comprises:
vi) from 3 to 10 ng/mL of recombinant angiopoietin-1 (ANG-1);
vii) from 9 to 40 ng/mL of recombinant hepatocyte growth factor (HGF);
viii) from 9 to 40 ng/mL of recombinant insulin; and
ix) from 1.5 to 5 ng/mL of recombinant vascular endothelial growth factor (VEGF).

5. The composition of claim 1, wherein said composition comprises:
i) from 1.5 to 3 ng/mL of recombinant FGF-7;
ii) from 12 to 25 ng/mL of recombinant follistatin;
iii) from 6 to 12 ng/mL of recombinant IL-6;
iv) from 45 to 100 ng/mL of recombinant TGFβ-1; and
v) from 6 to 12 ng/mL of recombinant uPAR.

6. The composition of claim 5, wherein said composition further comprises:
vi) from 5 to 10 ng/mL of recombinant ANG-1;
vii) from 18 to 40 ng/mL of recombinant HGF;
viii) from 18 to 40 ng/mL of recombinant insulin; and
ix) from 3 to 5 ng/mL of recombinant VEGF.

7. The composition of claim 1, wherein said composition comprises:
i) from 1.5 to 2.5 ng/mL of recombinant FGF-7;
ii) from 12 to 20 ng/mL of recombinant follistatin;
iii) from 6 to 10 ng/mL of recombinant IL-6;
iv) from 45 to 80 ng/mL of recombinant TGFβ-1; and
v) from 6 to 10 ng/mL of recombinant uPAR.

8. The composition of claim 7, wherein said composition further comprises:
vi) from 5 to 8 ng/mL of recombinant ANG-1;
vii) from 18 to 30 ng/mL of recombinant HGF;
viii) from 18 to 30 ng/mL of recombinant insulin; and
ix) from 3 to 4 ng/mL of recombinant VEGF.

9. The composition of claim 1, wherein the carrier comprises a media or saline, and optionally wherein the carrier is sterile.

10. (canceled)

11. The composition of claim 1, wherein the composition is frozen (e.g., freeze-dried).

12. The composition of claim 1, wherein the carrier is a hydrogel.

13. The composition of claim 12, wherein the hydrogel comprises alginate (e.g., crosslinked alginate), gelatin, or a combination thereof (e.g., a combination thereof at a ratio of from 2:1 or 1.5:1, to 1:1.5 or 1:2 by weight) (e.g., 1, 2 or 3% by weight of an alginate and gelatin mixture).

14. The composition of claim 1, wherein the carrier is a foam.

15. The composition of claim 14, wherein the foam comprises alginate (e.g., crosslinked alginate), gelatin, or a combination thereof (e.g., a combination thereof at a ratio of from 2:1 or 1.5:1, to 1:1.5 or 1:2 by weight) (e.g., 1, 2 or 3% by weight of an alginate and gelatin mixture).

16. The composition of claim 1, wherein the carrier comprises a cream or gel suitable for topical application to a skin wound.

17. The composition of claim 1, wherein the carrier is in the form of a patch or sheet.

18. The composition of claim 1, wherein the composition further comprises one or more pH-adjusting agents or buffers, softening agents, protein stabilizers, preservatives, or color additives.

19. A method for treating a wound, comprising applying the composition of claim 1 to said wound.

20. The method of claim 19, wherein said composition is applied to said wound in an amount effective to accelerate healing of the wound (e.g., accelerate wound closure, tissue formation, and/or re-epithelialization), wherein said composition is applied to said wound in an amount effective to accelerate angiogenesis of the wound, and/or wherein said composition is applied to said wound in an amount effective to reduce scarring (e.g., promote basket-weave collage network formation).

21.-22. (canceled)

23. The method of claim 19, wherein said wound is hypoxic.

24. The method of claim 19, wherein said wound is a burn wound.

25. The method of claim 19, wherein said wound is a full-thickness skin wound (e.g., full-thickness chronic skin wound).

26. A method for culturing cells in vitro, comprising culturing said cells in the presence of the composition of claim 1, optionally wherein said composition is provided in an amount effective to promote proliferation of the cells.

27. (canceled)

28. A method for cell delivery in vivo, comprising providing a composition of claim 1 which contains said cells, and delivering the composition to a subject in need thereof.
